(19) United States
(12) Patent Application Publication
(10) Pub. No.: US 2025/0122278 A1
(43) Pub. Date: Apr. 17, 2025

(54) ANTI-INTERLEUKIN-9 ANTIBODY AND USE THEREOF

(71) Applicant: GUANGDONG KEGUANDA PHARMACEUTICAL TECHNOLOGY CO., LTD., Guangzhou (CN)

(72) Inventors: Hung Yan Lau, Guangzhou (CN); Chin Yu Li, Guangzhou (CN)

(21) Appl. No.: 18/989,007

(22) Filed: Dec. 20, 2024

Related U.S. Application Data
(63) Continuation of application No. PCT/CN2023/100022, filed on Jun. 13, 2023.

(30) Foreign Application Priority Data
Jun. 20, 2022 (CN) 202210701293.0

(51) Int. Cl. C07K 16/24; A61K 39/00; A61K 39/395; A61K 45/06; A61P 11/00

(52) U.S. Cl. CPC C07K 16/244; A61K 39/3955; A61K 45/06; A61P 11/00; A61K 2039/505; C07K 2317/24; C07K 2317/76; C07K 2317/92

(57) ABSTRACT

The present disclosure provides an antibody specifically binding to interleukin-9 (IL-9), a nucleic acid encoding the antibody, a vector including the nucleic acid, a host cell including the nucleic acid or the vector, a pharmaceutical composition including the antibody, and a use of the antibody and the pharmaceutical composition.

### CROSS-REFERENCE TO RELATED APPLICATIONS

The present application is a continuation application of PCT application No. PCT/CN2023/100022 filed on Jun. 13, 2023, which claims the benefit of Chinese Patent Application No. 202210701293.0 filed on Jun. 20, 2022. The contents of all of the aforementioned applications are incorporated by reference herein in their entirety.

### TECHNICAL FIELD

The present disclosure relates to an antibody specifically binding to interleukin-9 (IL-9), and a use of the antibody and especially a use of the antibody in treatment of a respiratory disease, an autoimmune disease, and a hematologic cancer.

### REFERENCE TO SEQUENCE LISTING

The Sequence Listing XML file submitted via the USPTO Patent Center, with a file name of “Sequence listing_SCH-24109-USPT.xml”, a creation date of Dec. 20, 2024, and a size of 33,122 bytes, is part of the specification and is incorporated in its entirety by reference herein.

### BACKGROUND

Interleukin-9 (IL-9) is a monomeric glycosylated polypeptide of 28 kDa to 30 kDa. IL-9 is expressed by activated T cells and mast cells, and plays a key role in many antigen-induced reactions. IL-9 functions through interleukin-9 receptor (IL-9R). IL-9 can stimulate the proliferation of cells and prevent the apoptosis of cells. After IL-9R is activated, different pathways are activated through different signal transduction, such as Janus kinase (JAK) signaling pathway and signal transducer and activator of transcription (STAT) proteins (including STAT1, STAT3, and STAT5), such that IL-9 is associated with various biological processes. IL-9 can affect many effector cells, such as effector T cells, B cells, innate lymphoid cells, mast cells, polymorphonuclear cells, epithelial cells, and smooth muscle cells, and plays an important role in the inflammatory immune regulation.

The critical role of IL-9 in promoting cellular and humoral immune responses makes IL-9 become a potential target for therapeutic interventions. According to the previous reports, IL-9 plays an important role in the occurrence and progression of respiratory diseases, autoimmune diseases, and hematologic cancers, and the corresponding mechanisms may be related to the functions of IL-9 and IL-9-secreting T helper 9 cells.

The respiratory diseases can be caused by various pathogenic infections such as viral or bacterial infections and allergic reactions such as allergies. The autoimmune diseases are resulted from immune responses of the body to autoantibodies to cause the damage to tissues of the body itself. The respiratory diseases are mainly manifested as inflammations of respiratory tract tissues such as lungs. The autoimmune diseases can lead to non-infectious inflammatory changes in a variety of organs and tissues such as joints, synovium, soft tissues, and connective tissues throughout the body. There are many similarities between the respiratory diseases and the autoimmune diseases in terms of both aspects such as pathogens, pathogenesis, and pathological changes and aspects such as treatment pathways and drug action targets.

The autoimmune diseases are very difficult to treat, and require long-term therapy. The current therapies for inflammatory conditions include symptomatic medications and immunosuppressants. Drugs with analgesic and anti-inflammatory effects that are used in the symptomatic treatment include nonsteroidal anti-inflammatory drugs (NSAIDs). However, NSAIDs cannot alter the disease progression. In addition, NSAIDs often cause gastrointestinal side effects to affect the lower gastrointestinal tract, thereby causing perforation or exacerbating inflammatory bowel disease and causing nephrotoxicity. Corticosteroids are another class of drugs commonly used to control inflammation symptoms. Like NSAIDs, corticosteroids do not alter the natural progression of diseases. Thus, when the medication is discontinued, the clinical manifestations of active diseases often reappear. During on long-term corticosteroid therapy, patients undergo serious adverse reactions, which greatly limit the long-term use of corticosteroids. Immunosuppressants are also a common treatment option. Similarly, the long-term use of immunosuppressants can damage the immune system of a patient, such that the patient loses immunity to infections. Therefore, there is a need to develop new therapies for autoimmune conditions that can be applied for a long time.

In addition, in hematologic cancers such as Hodgkin's lymphoma, IL-9 is thought to act in a paracrine manner. IL-9 promotes the tumor development by stimulating and activating TH2 lymphocytes infiltrated in lymphoma and interacting with other cells constituting a tumor such as mast cells, monocytes, and cosinophils.

### SUMMARY

The present disclosure provides an antibody specifically binding to IL-9, a nucleic acid encoding the antibody, a vector including the nucleic acid, and a host cell including the nucleic acid or the vector. The present disclosure also discloses a pharmaceutical composition including the antibody, and a use of the antibody and the pharmaceutical composition and especially a use of the antibody in prevention or treatment of a respiratory disease, an autoimmune disease, and a hematologic cancer.

Accordingly, in an aspect, the present disclosure provides an antibody specifically binding to IL-9 or an antigen-binding fragment thereof, including a heavy chain variable region (VH) and a light chain variable region (VL), where

(i) the VH includes CDR-H1, CDR-H2, and CDR-H3 having amino acid sequences set forth in SEQ ID NOs: 1-3, respectively, and the VL includes CDR-L1, CDR-L2, and CDR-L3having an amino acid sequence set forth in SEQ ID NO: 5, an amino acid sequence SAS, and an amino acid sequence set forth in SEQ ID NO: 6, respectively; or

(ii) the VH includes CDR-H1, CDR-H2, and CDR-H3 having amino acid sequences set forth in SEQ ID NOs: 8-10, respectively, and the VL includes CDR-L1, CDR-L2, and CDR-L3 having an amino acid sequence set forth in SEQ ID NO: 12, an amino acid sequence AAT, and an amino acid sequence set forth in SEQ ID NO: 13, respectively; or

(iii) the VH includes CDR-H1, CDR-H2, and CDR-H3 having amino acid sequences set forth in SEQ ID NOs: 15-17, respectively, and the VL includes CDR-L1, CDR-L2, and CDR-L3 having an amino acid sequence set forth in SEQ ID NO: 19, an amino acid sequence YAS, and an amino acid sequence set forth in SEQ ID NO: 20, respectively; or

(iv) the VH includes CDR-H1, CDR-H2, and CDR-H3 having amino acid sequences set forth in SEQ ID NOs: 22-24, respectively, and the VL includes CDR-L1, CDR-L2, and CDR-L3 having an amino acid sequence set forth in SEQ ID NO: 12, an amino acid sequence AAT, and an amino acid sequence set forth in SEQ ID NO: 13, respectively.

In some embodiments of the antibody or the antigen-binding fragment thereof in the present disclosure,

(i) the VH includes an amino acid sequence having at least 80%, at least 85%, at least 90%, at least 95%, at least 98%, at least 99%, or 100% sequence identity to SEQ ID NO: 4, and the VL includes an amino acid sequence having at least 80%, at least 85%, at least 90%, at least 95%, at least 98%, at least 99%, or 100% sequence identity to SEQ ID NO: 7; or

(ii) the VH includes an amino acid sequence having at least 80%, at least 85%, at least 90%, at least 95%, at least 98%, at least 99%, or 100% sequence identity to SEQ ID NO: 11, and the VL includes an amino acid sequence having at least 80%, at least 85%, at least 90%, at least 95%, at least 98%, at least 99%, or 100% sequence identity to SEQ ID NO: 14; or

(iii) the VH includes an amino acid sequence having at least 80%, at least 85%, at least 90%, at least 95%, at least 98%, at least 99%, or 100% sequence identity to SEQ ID NO: 18, and the VL includes an amino acid sequence having at least 80%, at least 85%, at least 90%, at least 95%, at least 98%, at least 99%, or 100% sequence identity to SEQ ID NO: 21; or

(iv) the VH includes an amino acid sequence having at least 80%, at least 85%, at least 90%, at least 95%, at least 98%, at least 99%, or 100% sequence identity to SEQ ID NO: 25, and the VL includes an amino acid sequence having at least 80%, at least 85%, at least 90%, at least 95%, at least 98%, at least 99%, or 100% sequence identity to SEQ ID NO: 14.

In some embodiments,

(i) the VH includes an amino acid sequence set forth in SEQ ID NO: 4, and the VL includes an amino acid sequence set forth in SEQ ID NO: 7; or

(ii) the VH includes an amino acid sequence set forth in SEQ ID NO: 11, and the VL includes an amino acid sequence set forth in SEQ ID NO: 14; or

(iii) the VH includes an amino acid sequence set forth in SEQ ID NO: 18, and the VL includes an amino acid sequence set forth in SEQ ID NO: 21; or

(iv) the VH includes an amino acid sequence set forth in SEQ ID NO: 25, and the VL includes an amino acid sequence set forth in SEQ ID NO: 14.

In some embodiments of the antibody or the antigen-binding fragment thereof in the present disclosure, the VH includes an amino acid sequence having at least 80%, at least 85%, at least 90%, at least 95%, at least 98%, at least 99%, or 100% sequence identity to an amino acid sequence set forth in any one of SEQ ID NOs: 26-29, and the VL includes an amino acid sequence having at least 80%, at least 85%, at least 90%, at least 95%, at least 98%, at least 99%, or 100% sequence identity to an amino acid sequence set forth in any one of SEQ ID NOs: 30-33.

In some embodiments, the VH includes an amino acid sequence set forth in any one of SEQ ID NOs: 26-29, and the VL includes an amino acid sequence set forth in any one of SEQ ID NOs: 30-33.

In a preferred embodiment,

(i) the VH includes an amino acid sequence set forth in SEQ ID NO: 26, and the VL includes an amino acid sequence set forth in SEQ ID NO: 31; or

(ii) the VH includes an amino acid sequence set forth in SEQ ID NO: 26, and the VL includes an amino acid sequence set forth in SEQ ID NO: 33; or

(iii) the VH includes an amino acid sequence set forth in SEQ ID NO: 27, and the VL includes an amino acid sequence set forth in SEQ ID NO: 33; or

(iv) the VH includes an amino acid sequence set forth in SEQ ID NO: 28, and the VL includes an amino acid sequence set forth in SEQ ID NO: 33.

In some embodiments of the antibody or the antigen-binding fragment thereof in the present disclosure, the antibody can be selected from the group consisting of IgG, IgA, IgM, IgE, and IgD isotypes, such as IgG1, IgG2, IgG3, and IgG4 subtypes.

In some embodiments, the antibody can be selected from the group consisting of a murine antibody, a chimeric antibody, a humanized antibody, and a fully human antibody.

In some embodiments, the antigen-binding fragment can be selected from the group consisting of Fab, Fab′, F(ab′)₂, Fv, scFv, and ds-scFv.

In some embodiments, the antibody can be a bispecific antibody or a multispecific antibody.

In another aspect, the present disclosure provides a nucleic acid including a nucleotide sequence encoding the antibody or the antigen-binding fragment thereof of the present disclosure.

In yet another aspect, the present disclosure provides a vector including a nucleotide sequence encoding the antibody or the antigen-binding fragment thereof of the present disclosure.

In yet another aspect, the present disclosure provides a host cell including the nucleic acid of the present disclosure or the vector of the present disclosure.

In yet another aspect, the present disclosure provides a pharmaceutical composition, including: the antibody or the antigen-binding fragment thereof of the present disclosure, and a pharmaceutically acceptable carrier or excipient.

In some embodiments of the pharmaceutical composition in the present disclosure, the pharmaceutical composition further includes a second therapeutic agent. In some embodiments, the second therapeutic agent can be selected from the group consisting of an antibody, a chemotherapeutic agent, and a small molecule drug. In some embodiments, the second therapeutic agent can be selected from the group consisting of an antiviral agent, an antibacterial agent, an antifungal agent, an antiangiogenic agent, a TNF-α antagonist, an immunomodulator, an anticancer agent, a mast cell modulator, an anti-inflammatory agent, an antihistamine, and an active ingredient and extract of a common traditional Chinese medicine, and a combination thereof.

In yet another aspect, the present disclosure provides a method for preventing or treating a disease associated with abnormal expression of IL-9 in a subject, including administering a therapeutically effective amount of the antibody or the antigen-binding fragment thereof of the present disclosure or the pharmaceutical composition of the present disclosure to the subject.

In yet another aspect, the present disclosure provides a use of the antibody or the antigen-binding fragment thereof of the present disclosure or the pharmaceutical composition of the present disclosure in prevention or treatment of a disease associated with abnormal expression of IL-9 in a subject.

In yet another aspect, the present disclosure provides a use of the antibody or the antigen-binding fragment thereof of the present disclosure or the pharmaceutical composition of the present disclosure in preparation of a drug for preventing or treating a disease associated with abnormal expression of IL-9 in a subject.

In some embodiments of the above aspect of the present disclosure, the disease associated with abnormal expression of IL-9 can be a respiratory disease. In some embodiments, the respiratory disease can be selected from the group consisting of acute infectious pneumonia, hypersensitivity pneumonitis, bronchial asthma, chronic obstructive pulmonary disease, and pulmonary fibrosis.

In some embodiments, the disease associated with abnormal expression of IL-9 can be an autoimmune disease. In some embodiments, the autoimmune disease can be selected from the group consisting of systemic lupus erythematosus, rheumatoid arthritis, systemic vasculitis, scleroderma, dermatomyositis, mixed connective tissue disease, allergic rhinitis, allergic conjunctivitis, autoimmune hemolytic anemia, autoimmune thyroid disease, ulcerative colitis, Sjogren's syndrome, psoriatic arthritis, multiple sclerosis, chronic lymphocytic thyroiditis, insulin-dependent diabetes mellitus, myasthenia gravis, pernicious anemia with chronic atrophic gastritis, pulmonary hemorrhage-nephritic syndrome, pemphigus vulgaris, pemphigoid, multiple cerebrospinal sclerosis, acute idiopathic polyneuritis, and autoimmune adrenal insufficiency.

In some embodiments, the disease associated with abnormal expression of IL-9 can be a hematologic cancer, such as leukemia, malignant plasma cell neoplasm, or lymphoma. In some embodiments, the hematologic cancer can be selected from the group consisting of chronic myeloid leukemia, acute myeloid leukemia, chronic lymphocytic leukemia, acute lymphocytic leukemia, multiple myeloma, monoclonal gammopathy of undetermined significance (MGUS), non-Hodgkin's lymphoma, and Hodgkin's lymphoma.

In some embodiments of the above aspect of the present disclosure, the method further includes administering a second therapeutic agent to the subject. In some embodiments, the antibody or the antigen-binding fragment thereof of the present disclosure or the pharmaceutical composition of the present disclosure is administered in combination with the second therapeutic agent.

In some embodiments, the second therapeutic agent can be selected from the group consisting of an antibody, a chemotherapeutic agent, and a small molecule drug. In some embodiments, the second therapeutic agent can be selected from the group consisting of an antiviral agent, an antibacterial agent, an antifungal agent, an antiangiogenic agent, a TNF-α antagonist, an immunomodulator, an anticancer agent, a mast cell modulator, an anti-inflammatory agent, an antihistamine, and an active ingredient and extract of a common traditional Chinese medicine, and a combination thereof.

### BRIEF DESCRIPTION OF THE DRAWINGS

The comprehension of the features and advantages of the present application can be acquired with reference to the following detailed description and accompanying drawings describing the exemplary embodiments utilizing the principle of the present application.

FIG. 1 shows sodium dodecyl sulfate-polyacrylamide gel electrophoresis (SDS-PAGE) assay results of Fc-IL-9 and IL-9-His recombinant proteins, where a lane M is for a protein marker, the lane 9 is for the Fc-IL-9 recombinant protein, and the lane 1 is for the IL-9-His recombinant protein;

FIG. 2 shows fluorescence-activated cell sorting (FACS) results for preparation of a CHO-S-IL-9 cell line (i.e., a CHO-S cell expressing IL-9 on the cell membrane), where negative control (NC) is phosphate buffered saline (PBS) and control cells are CHO-S;

FIG. 3 shows flow cytometry results of the blocking activity of purified antibodies, where a positive control (PC) is an IL-9 positive antibody 7F3com (as shown in the patent CN1809383A);

FIG. 4 shows schematic structural diagrams of pcDNA3.4-IgG1 and pcDNA3.4-IgKc expression vectors;

FIG. 5 shows flow cytometry results of the blocking activity and EC₅₀ values of chimeric antibodies, where PC is 7F3com;

FIG. 6A to FIG. 6B show enzyme-linked immunosorbent assay (ELISA) results (FIG. 6A) and flow cytometry results of the blocking activity (FIG. 6B) for humanized antibodies, where in ELISA, controls are the 38-D8-C7-G9 chimeric antibody (CA1) and 7F3com; and in flow cytometry, the control is the 38-D8-C7-G9 chimeric antibody (CA1);

FIG. 7A to FIG. 7C show affinity results of humanized antibodies determined by ForteBio (bio-layer interferometry (BLI)), where FIG. 7A is for the 38-D8-C7-G9 chimeric antibody (CA1), FIG. 7B is for a humanized VH1−VL2 antibody, and FIG. 7C is for a humanized VH1−VL4 antibody;

FIG. 8A to FIG. 8B show changes in body weights of a bleomycin mouse model group (FIG. 8A) and a silica mouse model group (FIG. 8B) over time;

FIG. 9A to FIG. 9B show Hematoxylin and Eosin (H&E) staining (FIG. 9A) and inflammation scoring (FIG. 9B) results for the bleomycin mouse model group (**=P<0.01 and ***=P<0.001);

FIG. 10A to FIG. 10B show Masson staining (FIG. 10A) and fibrosis scoring (FIG. 10B) results for the bleomycin mouse model group (ns: not significant, *=P<0.05, and ***=P<0.001);

FIG. 11A to FIG. 11B show H&E staining (FIG. 11A) and inflammation scoring (FIG. 11B) results for the silica mouse model group (*=P<0.05, **=P<0.01, and ***=P<0.001); and

FIG. 12A to FIG. 12B show Masson staining (FIG. 12A) and fibrosis scoring (FIG. 12B) results for the silica mouse model group (*=P<0.05 and ***=P<0.001).

### DETAILED DESCRIPTION

The present disclosure provides an antibody with high affinity for IL-9 or an antigen-binding fragment thereof, which can be used for treating a respiratory disease (such as acute infectious pneumonia, hypersensitivity pneumonitis, bronchial asthma, chronic obstructive pulmonary disease, and pulmonary fibrosis, idiopathic pulmonary fibrosis, etc. resulting from various factors), an autoimmune disease (such as systemic lupus erythematosus, rheumatoid arthritis, systemic vasculitis, scleroderma, dermatomyositis, mixed connective tissue disease, autoimmune hemolytic anemia, autoimmune thyroid disease, ulcerative colitis, Sjogren's syndrome, psoriatic arthritis, multiple sclerosis, chronic lymphocytic thyroiditis, insulin-dependent diabetes mellitus, myasthenia gravis, pernicious anemia with chronic atrophic gastritis, pulmonary hemorrhage-nephritic syndrome, pemphigus vulgaris, pemphigoid, multiple cerebrospinal sclerosis, acute idiopathic polyneuritis, and autoimmune adrenal insufficiency), and a hematologic cancer (such as leukemia (such as chronic myeloid leukemia, acute myeloid leukemia, chronic lymphocytic leukemia, and acute lymphocytic leukemia), malignant plasma cell neoplasm (such as multiple myeloma and MGUS), or lymphoma (such as non-Hodgkin's lymphoma and Hodgkin's lymphoma) associated with IL-9 expression and/or activity.

Any aspect or embodiment described herein may be combined with any other aspect or embodiment disclosed herein. Although the present disclosure has been described in conjunction with the detailed description of the present disclosure, the above description is intended to illustrate rather than limit the scope of the present disclosure. The scope of the present disclosure is defined by the scope of the appended claims. Other aspects, advantages, and modifications are also within the scope of the appended claims. The patents and scientific literature mentioned herein provide the knowledge accessible to those skilled in the art. All Chinese patents and published or unpublished Chinese patent applications cited herein are incorporated herein by reference. All published foreign patents and patent applications cited herein are hereby incorporated herein by reference. All other published references, dictionaries, documents, manuscripts, and scientific literature cited herein are hereby incorporated herein by reference. From the following detailed description including embodiments and claims, other features and advantages of the present disclosure will be apparent.

In order to make it easy to understand the present disclosure, some terms are first defined below. Additional definitions of the following terms and other terms are set forth throughout the specification.

Unless otherwise defined, all technical and scientific terms used herein have the same meanings as commonly understood by those of ordinary skill in the art.

It should be noted that, as used herein and in the appended claims, the singular forms “a”, “an”, and “the” include plural references, unless otherwise specified clearly in the context. Thus, the terms “a”, “an”, “a or a plurality of”, and “at least one” can be used interchangeably. Similarly, the terms “include”, “comprise”, and “have” can be used interchangeably.

When the terms “include”, “comprise”, and “have” are used herein and in the appended claims, other elements are not excluded. For the purpose of the present application, the term “composed of . . . ” is considered as a preferred embodiment of the term “include”.

The present disclosure provides an antibody specifically binding to IL-9 or an antigen-binding fragment thereof, including VH and VL, where

(i) the VH includes CDR-H1, CDR-H2, and CDR-H3 having amino acid sequences set forth in SEQ ID NOs: 1-3, respectively, and the VL includes CDR-L1, CDR-L2, and CDR-L3 having an amino acid sequence set forth in SEQ ID NO: 5, an amino acid sequence SAS, and an amino acid sequence set forth in SEQ ID NO: 6, respectively; or

(ii) the VH includes CDR-H1, CDR-H2, and CDR-H3 having amino acid sequences set forth in SEQ ID NOs: 8-10, respectively, and the VL includes CDR-L1, CDR-L2, and CDR-L3 having an amino acid sequence set forth in SEQ ID NO: 12, an amino acid sequence AAT, and an amino acid sequence set forth in SEQ ID NO: 13, respectively; or

(iii) the VH includes CDR-H1, CDR-H2, and CDR-H3 having amino acid sequences set forth in SEQ ID NOs: 15-17, respectively, and the VL includes CDR-L1, CDR-L2, and CDR-L3 having an amino acid sequence set forth in SEQ ID NO: 19, an amino acid sequence YAS, and an amino acid sequence set forth in SEQ ID NO: 20, respectively; or

(iv) the VH includes CDR-H1, CDR-H2, and CDR-H3 having amino acid sequences set forth in SEQ ID NOs: 22-24, respectively, and the VL includes CDR-L1, CDR-L2, and CDR-L3 having an amino acid sequence set forth in SEQ ID NO: 12, an amino acid sequence AAT, and an amino acid sequence set forth in SEQ ID NO: 13, respectively.

As used in the present disclosure, the term “antibody” refers to an immunoglobulin molecule with an ability of specifically binding to a specific antigen. An antibody typically includes a variable region and a constant region in each heavy chain and each light chain. Variable regions of heavy and light chains of an antibody each include a binding domain interacting with an antigen. Constant regions of an antibody can mediate the binding of an immunoglobulin to a host tissue or factor. The host tissue or factor includes various cells (such as effector cells) of an immune system and components of a complement system. Thus, most antibodies have VH and VL that together constitute an antibody portion to bind to an antigen.

VH or VL includes framework regions (FRs) separated by three complementarity-determining regions (CDRs). FR is generated to adjust CDR for specifically binding to an epitope. CDRs include amino acid residues in an antibody that are primarily responsible for antigen binding. From an amino terminus to a carboxyl terminus, VH and VL domains each include the following FRs and CDRs: FR1, CDR1, FR2, CDR2, FR3, CDR3, and FR4. CDR1, CDR2, and CDR3 of a VH domain are also referred to as CDR-H1, CDR-H2, and CDR-H3 herein, respectively. CDR1, CDR2, and CDR3 of a VL domain are also referred to as CDR-L1, CDR-L2, and CDR-L3 herein, respectively.

The amino acid assignment for each VH and VL domain is based on any conventional definition of CDR. The conventional definition includes a Kabat definition (Kabat, Sequences of Proteins of Immunological Interest (National Institutes of Health, Bethesda, MD, 1987 and 1991)), a Chothia definition (Chothia & Lesk, J. Mol. Biol. 196: 901-917, 1987; Chothia et al., Nature 342: 878-883, 1989), a union of Chothia and Kabat definitions, an AbM definition adopted by antibody modeling software of Oxford Molecular, and a CONTACT definition of Martin et al. (www.bioinfo.org.uk/abs). The Kabat provides a widely-used numbering convention (Kabat numbering system), where corresponding residues of different heavy chains or different light chains are given same numbers. The present disclosure may adopt CDRs defined by any of these numbering systems. In some embodiments, CDRs defined by Kabat are adopted. In some embodiments, CDRs defined by Chothia are adopted. In some embodiments, CDRs defined by Kabat are adopted. In some embodiments, CDRs defined by a combination of Chothia and Kabat are adopted. In some embodiments, CDRs defined by AbM are adopted. In some embodiments, CDRs defined by CONTACT are adopted.

As used in the present disclosure, the term “binding” or “specifically binding” refers to a non-random binding reaction between two molecules, such as a non-random binding reaction between an antibody and a target antigen thereof. The binding specificity of an antibody can be determined based on affinity and/or binding activity. The affinity is usually expressed by an equilibrium constant (KD) for the dissociation of an antigen from an antibody, and is a measure of a binding strength between an epitope and an antigen-binding site of an antibody. The smaller the KD value, the stronger the binding strength between an epitope and an antibody. Alternatively, the affinity can also be expressed as an affinity constant (KA), which is 1/KD. KD can be calculated from a quotient of K_{off}/Kₒₙ, and KA can be calculated from a quotient of Kₒₙ/K_{off}. Kₒₙ refers to, for example, a binding rate constant of an antibody to an antigen, and K_{off} refers to, for example, a dissociation rate constant of an antibody from an antigen. Kₒₙ and K_{off} can be determined by techniques known to those of ordinary skill in the art (such as BIAcore® or KinExA).

Typically, an antibody will bind to an antibody with a dissociation constant (KD) of 10⁻⁵ to 10⁻¹² M or less, preferably 10⁻⁷ to 10⁻¹² M or less, and more preferably 10⁻⁸ to 10⁻¹² M, and/or with a binding affinity of at least 10⁷ M⁻¹, preferably at least 10⁸ M⁻¹, and more preferably at least 10⁹ M⁻¹, such as at least 10¹² M⁻¹. Any KD value greater than 10⁻⁴ M is generally considered to indicate non-specific binding. The specific binding of an antibody to an antigen or epitope can be determined by any suitable known method, including, for example, Scatchard analysis and/or competitive binding assays, such as radioimmunoassays (RIAs), enzyme immunoassays (EIAs), and sandwich and competitive assays and different variations known in the art.

In some embodiments of the antibody or the antigen-binding fragment thereof in the present disclosure,

(i) the VH includes an amino acid sequence having at least 80%, at least 85%, at least 90%, at least 95%, at least 98%, at least 99%, or 100% sequence identity to SEQ ID NO: 4, and the VL includes an amino acid sequence having at least 80%, at least 85%, at least 90%, at least 95%, at least 98%, at least 99%, or 100% sequence identity to SEQ ID NO: 7; or

(ii) the VH includes an amino acid sequence having at least 80%, at least 85%, at least 90%, at least 95%, at least 98%, at least 99%, or 100% sequence identity to SEQ ID NO: 11, and the VL includes an amino acid sequence having at least 80%, at least 85%, at least 90%, at least 95%, at least 98%, at least 99%, or 100% sequence identity to SEQ ID NO: 14; or

(iii) the VH includes an amino acid sequence having at least 80%, at least 85%, at least 90%, at least 95%, at least 98%, at least 99%, or 100% sequence identity to SEQ ID NO: 18, and the VL includes an amino acid sequence having at least 80%, at least 85%, at least 90%, at least 95%, at least 98%, at least 99%, or 100% sequence identity to SEQ ID NO: 21; or

(iv) the VH includes an amino acid sequence having at least 80%, at least 85%, at least 90%, at least 95%, at least 98%, at least 99%, or 100% sequence identity to SEQ ID NO: 25, and the VL includes an amino acid sequence having at least 80%, at least 85%, at least 90%, at least 95%, at least 98%, at least 99%, or 100% sequence identity to SEQ ID NO: 14.

In some embodiments of the antibody or the antigen-binding fragment thereof in the present disclosure, the VH includes an amino acid sequence having at least 80%, at least 85%, at least 90%, at least 95%, at least 98%, at least 99%, or 100% sequence identity to an amino acid sequence set forth in any one of SEQ ID NOs: 26-29, and the VL includes an amino acid sequence having at least 80%, at least 85%, at least 90%, at least 95%, at least 98%, at least 99%, or 100% sequence identity to an amino acid sequence set forth in any one of SEQ ID NOs: 30-33.

In a preferred embodiment,

(i) the VH includes an amino acid sequence set forth in SEQ ID NO: 26, and the VL includes an amino acid sequence set forth in SEQ ID NO: 31; or

(ii) the VH includes an amino acid sequence set forth in SEQ ID NO: 26, and the VL includes an amino acid sequence set forth in SEQ ID NO: 33; or

(iii) the VH includes an amino acid sequence set forth in SEQ ID NO: 27, and the VL includes an amino acid sequence set forth in SEQ ID NO: 33; or

(iv) the VH includes an amino acid sequence set forth in SEQ ID NO: 28, and the VL includes an amino acid sequence set forth in SEQ ID NO: 33.

As used herein, the “sequence identity” refers to an identity degree between any given query sequence and a subject sequence. Those skilled in the art will easily understand how to determine an identity between two peptides (such as an unmodified peptide and a peptide variant. For example, an identity between two sequences can be calculated after the two sequences are aligned to reach the highest identity level, for example, gaps can be introduced. An identity can also be calculated by the published algorithms. Non-restrictive examples of such mathematical algorithms include an algorithm in Myers and Miller (1988) CABIOS 4: 11-17, a local homology algorithm in Smith et al. (1981) Adv. Appl. Math. 2: 482, a homology alignment algorithm in Needleman and Wunsch (1970) J. Mol. Biol. 48: 443-453, modified forms of a method of searching for a homology in Pearson and Lipman (1988) Proc. Natl. Acad. Sci. 85: 2444-2448 and an algorithm in Karlin and Altschul (1990) Proc. Natl. Acad. Sci. USA 87: 2264,and an algorithm recorded in Karlin and Altschul (1993) Proc. Natl. Acad. Sci. USA 90: 5873-5877. The sequence comparison (namely, alignment) for determining a sequence identity can be implemented with a program based on such a mathematical algorithm. The program can be properly executed by a computer. Examples of such a program include, but are not limited to, CLUSTAL and ALIGN programs (version 2.0) of a PC/Gene package and GAP, BESTFIT, BLAST, FASTA, and TFASTA of a Wisconsin genetics software package. For example, these programs can be implemented with initial parameters to conduct an alignment.

In addition, when a sequence identity between two amino acid sequences is determined, technicians may consider the so-called conservative amino acid substitution. The conservative amino acid substitution can often be described as an amino acid substitution in which an amino acid residue is substituted with another amino acid residue having a similar chemical structure, which has little or no impact on a function, activity, or other biological properties of a polypeptide. Such a conservative amino acid substitution is well known in the art.

The conservative substitution preferably refers to a substitution in which an amino acid residue from the following groups (a) to (e) is substituted with another amino acid residue from the same group as the amino acid residue: (a) small aliphatic, non-polar, or weakly-polar residues: Ala, Ser, Thr, Pro, and Gly; (b) polar and negatively-charged residues and (uncharged) amides thereof: Asp, Asn, Glu, and Gln; (c) polar and positively-charged residues: His, Arg, and Lys; (d) large aliphatic and non-polar residues: Met, Leu, He, Val, and Cys; and (c) aromatic residues: Phe, Tyr, and Trp.

Particularly preferred conservative substitutions are as follows: Ala to Gly or Ser; Arg to Lys; Asn to Gln or His; Asp to Glu; Cys to Ser; Gln to Asn; Glu to Asp; Gly to Ala or Pro; His to Asn or Gln; Ile to Leu or Val; Leu to Ile or Val; Lys to Arg, Gln, or Glu; Met to Leu, Tyr, or Ile; Phe to Met, Leu, or Tyr; Ser to Thr; Thr to Ser; Trp to Tyr; Tyr to Trp; and/or Phe to Val, Ile, or Leu.

In some embodiments, the antibody and the antigen-binding fragment thereof in the present disclosure include variants, and the variants may include one or more amino acid modifications. A single variant can include a substitution, a deletion, an insertion, an addition, or any combination thereof, as long as the variant can specifically bind to an antigen. Methods and techniques for preparing the variants are well known to those skilled in the art.

In some embodiments, the antibody can be selected from the group consisting of IgG, IgA, IgM, IgE, and IgD isotypes, such as IgG1, IgG2, IgG3, and IgG4 subtypes. In some embodiments, the antibody can be selected from the group consisting of a murine antibody, a chimeric antibody, a humanized antibody, and a fully human antibody. In some embodiments, the antigen-binding fragment can be selected from the group consisting of Fab, Fab′, F(ab′)₂, Fv, scFv, and ds-scFv. In some embodiments, the antibody can be a bispecific antibody or a multispecific antibody.

The term “antibody” used in the present disclosure should be understood in its broadest sense, and includes a monoclonal antibody (including a full-length monoclonal antibody), a polyclonal antibody, an antibody fragment, and a multispecific antibody with at least two antigen-binding regions (such as a bispecific antibody). The antibody may include an additional modification, such as a non-natural amino acid, a mutation in a Fc region, and a mutation at a glycosylation site. The antibody also includes an antibody with a post-translational modification (PTM), a fusion protein including an antigen-binding site of an antibody, and an immunoglobulin molecule including any other modification for an antigen-recognition site, as long as these antibodies exhibit an expected biological activity.

In some embodiments, the antibody includes a chimeric antibody, such as a humanized form of a murine antibody. Such a humanized antibody can be prepared by a known technique, and exhibits the advantage of reducing the immunogenicity when administered to the human. In an embodiment, the humanized antibody includes a variable region of a murine antibody (or only an antigen-binding site thereof) and a constant region derived from a human antibody. Alternatively, a humanized antibody fragment can include an antigen-binding site of a murine antibody and a variable region fragment derived from a human antibody (lacking an antigen-binding site). Processes for producing chimeric antibodies and further engineered antibodies include those processes described in the following: Riechmann et al. (Nature 332: 323, 1988); Liu et al. (PNAS 84: 3439, 1987); Larrick et al. (Bio/Technology 7: 934, 1989); and Winter and Harris (TIPS 14: 139, May 1993). Processes for transgenic production of antibodies can be found in GB 2,272,440 and U.S. Pat. Nos. 5,569,825 and 5,545,806.

Antibodies (such as chimeric and humanized antibodies) that can be produced through genetic engineering each include both human and non-human moieties, and can be manufactured by standard recombinant DNA techniques. Such chimeric and humanized antibodies can be produced through genetic engineering by standard DNA techniques known in the art.

As used in the present disclosure, the term “antigen-binding fragment” of an antibody refers to one or more antibody fragments that maintain the ability to specifically bind to an antigen (such as IL-9). It has been proved that an antigen-binding function of an antibody can be implemented by a fragment of the full-length antibody. Examples of the antigen-binding fragment include: (i) a Fab fragment, which is a monovalent fragment including VH, VL, CH1, and CL domains; (ii) a F(ab′)₂ fragment, which is a bivalent fragment including two Fab fragments linked through a disulfide bond in a hinge region; (iii) a Fab′ fragment, which is essentially Fab but has a partial hinge region (as shown in FUNDAMENTAL IMMUNOLOGY (Paul ed., 3.sup.rd ed. 1993)); (iv) a Fd fragment, which includes VH and CH1 domains; (v) a Fd′ fragment, which includes VH and CH1 domains and one or more cysteine residues located at a C-terminus of a CH1 domain; (vi) a Fv fragment, which includes VH and VL domains of a single arm of an antibody; (vii) a dAb fragment (Ward et al. (1989) Nature 341: 544-546), which includes a VH domain; (viii) a separate CDR; and (ix) a nanobody, which is VH including a single variable domain and two constant domains. In addition, although the two domains of the Fv fragment, VH and VL, are encoded by separate genes, the two domains can be linked with a synthetic linker by a recombination method. The synthetic linker enables the generation of a single protein chain in which the VH and VL are paired to produce a monovalent molecule (known as single-chain Fv (scFv), see, for example, Bird et al. (1988) Science 242, 423-426 and Huston et al. (1988) Proc. Natl. Acad. Sci. USA 85, 5879-5883). Such a single-chain antibody is also intended to be covered by the term “antigen-binding fragment” of an antibody. In addition, the term includes a linear antibody with a pair of tandem Fd fragments (VH−CH1−VH−CH1), which forms an antigen-binding region together with a complementary light chain polypeptide and a modified version of any of the above fragments with an antigen-binding activity retained.

These antigen-binding fragments can be prepared by the conventional techniques known to those skilled in the art, and the utility of the fragments is screened in the same way as intact antibodies.

In an aspect, the present disclosure provides a nucleic acid including a nucleotide sequence encoding the antibody or the antigen-binding fragment thereof of the present disclosure.

The terms “nucleic acid”, “nucleotide”, and “nucleotide sequence” can be used interchangeably, and refer to oligomers and polymers of any length that are essentially made up of nucleotides (such as deoxyribonucleotides and/or ribonucleotides). The nucleic acid can include purine and/or pyrimidine bases and/or other natural bases (such as xanthine, inosine, and hypoxanthine), chemically or biochemically-modified (such as methylated) bases, and unnatural or derived nucleotide bases. A backbone of the nucleic acid can include a sugar and a phosphate group normally present in RNA or DNA, and/or one or more modified or substituted sugars and/or one or more modified or substituted phosphate groups. A modification for a phosphate group or a sugar can be introduced to improve the stability, the resistance to enzymatic degradation, or some other useful properties. The nucleic acid can be, for example, double-stranded, partially double-stranded, or single-stranded. When the nucleic acid is single-stranded, the nucleic acid can have a sense strand or an antisense strand. The nucleic acid can be circular or linear. As used herein, the term “nucleic acid” encompasses both DNA and RNA, including a genome, pre-mRNA, mRNA, cDNA, and a recombinant or synthetic nucleic acid carried by a vector. For the purpose of the use described herein, it should be understood that a polynucleotide can be modified by any method available in the art.

Those skilled in the art will understand that many different polynucleotides and nucleic acids can encode a same polypeptide due to the degeneracy in the genetic code. In addition, it should be understood that a nucleotide substitution not affecting a polypeptide sequence encoded by the polynucleotide described herein may conducted by technicians using a conventional technique to reflect the codon use of any specific host organism in which the polypeptide is to be expressed.

In another aspect, the present disclosure provides a vector including the nucleic acid of the present disclosure.

The term “vector” used herein refers to a nucleic acid delivery vehicle into which a nucleotide can be inserted. When a vector can make a protein encoded by an inserted nucleotide expressed, the vector is called an expression vector. A vector can be introduced into a host cell through a method such as transformation, transduction, or transfection, such that a genetic material element carried by the vector can be expressed in the host cell. Vectors are well known to those skilled in the art, and include but are not limited to: (1) a plasmid; (2) a phagemid; (3) a cosmid; (4) an artificial chromosome, such as a yeast artificial chromosome, a bacterial artificial chromosome, or a P1-derived artificial chromosome; (5) a phage, such as a λ phage or an M13 phage; and (6) an animal virus, such as a retrovirus, an adenovirus, an adeno-associated virus, a lentivirus, a herpes virus, a poxvirus, and a baculovirus. A vector may include a plurality of elements for controlling the expression, including but not limited to a promoter sequence, a transcription initiation sequence, an enhancer sequence, a selection element, and a reporter gene. In addition, a vector can include a replication origin.

A recombinant expression vector can be any suitable recombinant expression vector. Suitable vectors include vectors designed for propagation and amplification or for expression or for both, such as plasmids and viruses. For example, vectors can be selected from the pUC series (Fermentas Life Sciences, Glen Burnie, Md.), the pBluescript series (Stratagene, LaJolla, CA), the pET series (Novagen, Madison, Wis.), the pGEX series (Pharmacia Biotech, Uppsala, Sweden), and the pEX series (Clontech, Palo Alto, Calif.). Phage vectors such as λGT10, λGT11, λZapII (Stratagene), λEMBL4, and λNM1149 can also be adopted. Examples of plant expression vectors that can be used in the present disclosure include pBI01, pBI101.2, pBI101.3, pBI121, and pBIN19 (Clontech). Examples of animal expression vectors that can be used in the present disclosure include pcDNA, pEUK-Cl, pMAM, and pMAMneo (Clontech).

In another aspect, the present disclosure provides a host cell including the nucleic acid of the present disclosure or the vector of the present disclosure.

As used herein, the term “host cell” refers to a cell in which an expression vector has been introduced. Any cell can be adopted as a host cell for the nucleic acid or vector in the present disclosure. In some embodiments, the host cell can be a prokaryotic cell, a fungal cell, a yeast cell, or a higher eukaryotic cell such as a mammalian cell. Suitable prokaryotic cells include, but are not limited to, cubacteria, such as gram-negative or gram-positive organisms, such as Enterobacteriaceae, such as Escherichia, such as Escherichia coli (E. coli); Enterobacter; Erwinia; Klebsiella; Proteus; Salmonella, such as Salmonella typhimurium; Serratia, such as Serratia marcescens; Shigella; Bacillus, such as Bacillus subtilis (B. subtilis) and Bacillus licheniformis (B. licheniformis); Pseudomonas, such as Pseudomonas aeruginosa (P. aeruginosa); and Streptomyces. In some embodiments, the host cell is a human cell. In some embodiments, the host cell is an immune cell. In some embodiments, the host cell includes, for example, a CHO cell, such as a CHO-S cell and a CHO-KI cell, or an HEK293 cell, such as HEK293A, HEK293T, and HEK293FS.

In yet another aspect, the present disclosure provides a pharmaceutical composition, including: the antibody or the antigen-binding fragment thereof of the present disclosure, and a pharmaceutically acceptable carrier or excipient.

The term “pharmaceutically acceptable” means that the carrier or adjuvant is compatible with other components of the composition and is not substantially toxic to a recipient, and/or the carrier or adjuvant is approved or can be used for inclusion in the pharmaceutical composition administered parenterally to the human.

In some embodiments, the carrier or excipient used with the composition of the present disclosure includes, but is not limited to, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monocthyl ether, and a surfactant polyoxyethylene-sorbitan monooleate.

In some embodiments of the pharmaceutical composition in the present disclosure, the pharmaceutical composition further includes a second therapeutic agent. In some embodiments, the second therapeutic agent can be selected from the group consisting of an antibody, a chemotherapeutic agent, and a small molecule drug.

In some embodiments, the second therapeutic agent may be selected from the group consisting of an antiviral agent (such as zidovudine, acyclovir, ganciclovir, vidarabine, idoxuridine, trifluridine, virazole, foscanet, amantadine, rimantadine, Saquinavir, indinavir, ritonavir, and interferon alfa), an antibacterial agent (such as penicillin, cephalosporin, imipenem, aztreonam, vancomycin, cycloserine, bacitracin, chloramphenicol, crythromycin, clindamycin, tetracycline, streptomycin, tobramycin, gentamicin, amikacin, kanamycin, neomycin, spectinomycin, trimethoprim, norfloxacin, rifampicin, polymyxin, amphotericin B, nystatin, ketoconazole, isoniazid, metronidazole, and pentamidinc), an antifungal agent (such as pyrrole, imidazole, triazole, itraconazole, polyene, amphotericin B, fluconazole, flucytosine, ketoconazole, caspofungin acetate, and voriconazole), an antiangiogenic agent, a TNF-α antagonist, an immunomodulator (such as methotrexate, leflunomide, cyclophosphamide, Cytoxan, imuran, cyclosporine A, dimethylamine tetracycline, and azathioprinc), an anticancer agent (including but not limited to a peptide, a polypeptide, a fusion protein, a nucleic acid, a small molecule, a mimic, a synthetic drug, an inorganic molecule, and an organic molecule), a mast cell modulator, an anti-inflammatory agent (such as adrenocortical hormones, corticosteroids (such as betamethasone, budesonide, flunisolide, fluticasone, triamcinolone, methylprednisolone, prednisolone, prednisone, and hydrocortisone), glucocorticoids, steroids, nonsteroidal anti-inflammatory drugs (such as aspirin, ibuprofen, diclofenac sodium, and COX-2 inhibitors), and leukotriene antagonists (such as montelukast, methylxanthine, zafirlukast, and zileuton), β2 agonists (such as salbutamol, biterol, fenoterol, isoetharine, orciprenaline, pirbuterol, salbutamol, terbutaline, formoterol, salmeterol, and salbutamol, terbutaline)), vitaxin, siplizumab, an antihistamine (such as diphenhydramine, meclastine, pyribenzamine, brompheniramine, chlorpheniramine, triprolidine, promethazine, hydroxyzine, astemizole, azatadine, cetirizine, cyproheptadine, loratadine, and fexofenadine), an anti-EphA2 antibody, and an active ingredient and extract of a common traditional Chinese medicine (such as anti-inflammatory and anti-cancer active small molecules and polysaccharide extracts (such as ginseng polysaccharides, dendrobium polysaccharides, Poria cocos polysaccharides, Ganoderma lucidum polysaccharides, Cordyceps polysaccharides, wolfberry polysaccharides, and Tremella fuciformis polysaccharides), and any combination thereof.

In another aspect, the present disclosure provides a method for preventing or treating a disease such as a disease associated with abnormal expression of IL-9 in a subject, including: a therapeutically effective amount of the antibody or the antigen-binding fragment thereof of the present disclosure or the pharmaceutical composition of the present disclosure is administered to the subject. In yet another aspect, the present disclosure provides a use of the antibody or the antigen-binding fragment thereof of the present disclosure or the pharmaceutical composition of the present disclosure in preparation of a drug for preventing or treating a disease such as a disease associated with abnormal expression of IL-9 in a subject.

As used herein, the term “prevention” means that a therapeutically effective amount of the antibody or the antigen-binding fragment thereof or the pharmaceutical composition of the present disclosure is administered to a subject to protect the subject from developing a disease associated with abnormal expression of IL-9. When the term “prevention” used herein is related to a given treatment for a given condition (such as the prevention of a disease associated with abnormal expression of IL-9), it means that a subject undergoing the treatment does not develop a clinically observable level of the condition at all, or develops more slowly and/or develops to a milder degree than a subject not undergoing the treatment. The term is not merely limited to situations in which a subject does not experience any condition. For example, if a treatment is given during a period when a patient is exposed to a stimulus expected to cause a manifestation of a given condition, and the treatment makes the subject experience fewer and/or milder condition symptoms than an expected outcome of no treatment, then the treatment can be considered to have prevented the condition.

As used herein, the term “treatment” refers to a method for acquiring a beneficial or desired outcome (including a clinical outcome). For the purpose of the present application, a beneficial or desired clinical outcome includes, but is not limited to, one or more of the following: alleviating one or more symptoms caused by a disease, reducing the severity of a disease, stabilizing a disease (for example, preventing or delaying the exacerbation of a disease), preventing or delaying the spread of a disease, preventing or delaying the recurrence of a disease, delaying or slowing the progression of a disease, improving a state of a disease, providing the remission (partial or complete) of a disease, reducing a dose for one or more other drugs required to treat a disease, delaying the progression of a disease, improving a quality of life, increasing a body weight, and/or prolonging a survival period. The method of the present application conceives any one or more of these aspects for the treatment.

In some embodiments, the treatment can be given after one or more symptoms have already appeared. In other embodiments, the treatment can be given in the absence of symptoms. For example, the treatment may be given to a susceptible individual before symptom onset, or may be given in combination with another therapeutic agent (for example, based on a symptom history, based on genetic or other predisposing factors, a disease therapy, or any combination thereof). The treatment can also be continued after symptoms have disappeared, for example, to prevent or delay the recurrence of the symptoms.

As used herein, the term “administration” refers to the introduction of an antibody or a composition into a subject, and includes the parallel or sequential introduction of antibodies or compositions. The term “administration” can refer to, for example, treatments, pharmacokinetics, diagnosis, research, placebos, and experimental methods. The term “administration” also covers in vitro and ex vivo treatments. A composition or an agent can be introduced into a subject through any suitable route. Examples of an appropriate method for administering the antibody or composition of the present disclosure include oral administration, intradermal injection, subcutaneous injection, intramuscular injection, intraosseous injection, intraperitoneal injection, and intravenous injection, and systemic or topical administration to a vicinity of a target site, but are not limited thereto. The administration includes both administration by oneself and administration by others. The administration can be conducted through any suitable route. The appropriate administration route allows a composition or an agent to exert an expected function. For example, if the appropriate route is intravenous injection, the antibody or the composition is administered by introducing the antibody or the composition into a vein of the subject.

In some embodiments of the present disclosure, the antibody or the composition of the present disclosure is packaged with or stored in a device for administration. A device for an injectable preparation includes, but is not limited to, an injection port, an automatic injector, an injection pump, and an injection pen. A device for a nebulized or powdered preparation includes, but is not limited to, an inhaler, an insufflator, and an aspirator. Therefore, the present disclosure includes a use of an administration device carrying the antibody or composition of the present disclosure in treatment or prevention of one or more of the conditions described herein.

The subject expected to be administered includes, but is not limited to, a human (namely, a male or female of any age group, such as a pediatric subject (such as an infant, a child, and an adolescent) or an adult subject (such as a youth, a middle-aged person, or the elderly)) and/or another non-human animal, such as a mammal (such as a primate).

In some embodiments of the method or the use of the present disclosure, the disease can be selected from the group consisting of a respiratory disease, an autoimmune disease, and a hematologic cancer (such as leukemia, malignant plasma cell neoplasm, or lymphoma). In some embodiments, the disease can be selected from the group consisting of acute infectious pneumonia, hypersensitivity pneumonitis, bronchial asthma, chronic obstructive pulmonary disease, pulmonary fibrosis (such as pulmonary fibrosis and idiopathic pulmonary fibrosis resulting from various factors), systemic lupus erythematosus, rheumatoid arthritis, systemic vasculitis, scleroderma, dermatomyositis, mixed connective tissue disease, allergic rhinitis, allergic conjunctivitis, autoimmune hemolytic anemia, autoimmune thyroid disease, ulcerative colitis, Sjogren's syndrome, psoriatic arthritis, multiple sclerosis, chronic lymphocytic thyroiditis, insulin-dependent diabetes mellitus, myasthenia gravis, pernicious anemia with chronic atrophic gastritis, pulmonary hemorrhage-nephritic syndrome, pemphigus vulgaris, pemphigoid, multiple cerebrospinal sclerosis, acute idiopathic polyneuritis, autoimmune adrenal insufficiency, chronic myeloid leukemia, acute myeloid leukemia, chronic lymphocytic leukemia, acute lymphocytic leukemia, multiple myeloma, MGUS, non-Hodgkin's lymphoma, and Hodgkin's lymphoma.

In some embodiments of the method or the use of the present disclosure, the prevention or treatment of the disease in the subject further includes: a second therapeutic agent is administered to the subject.

In some embodiments, the second therapeutic agent may be selected from the group consisting of an antiviral agent (such as zidovudine, acyclovir, ganciclovir, vidarabine, idoxuridine, trifluridine, virazole, foscanet, amantadine, rimantadine, Saquinavir, indinavir, ritonavir, and interferon alfa), an antibacterial agent (such as penicillin, cephalosporin, imipenem, aztreonam, vancomycin, cycloserine, bacitracin, chloramphenicol, crythromycin, clindamycin, tetracycline, streptomycin, tobramycin, gentamicin, amikacin, kanamycin, neomycin, spectinomycin, trimethoprim, norfloxacin, rifampicin, polymyxin, amphotericin B, nystatin, ketoconazole, isoniazid, metronidazole, and pentamidinc), an antifungal agent (such as pyrrole, imidazole, triazole, itraconazole, polyene, amphotericin B, fluconazole, flucytosine, ketoconazole, caspofungin acetate, and voriconazole), an antiangiogenic agent, a TNF-α antagonist, an immunomodulator (such as methotrexate, leflunomide, cyclophosphamide, Cytoxan, imuran, cyclosporine A, dimethylamine tetracycline, and azathioprine), an anticancer agent (including but not limited to a peptide, a polypeptide, a fusion protein, a nucleic acid, a small molecule, a mimic, a synthetic drug, an inorganic molecule, and an organic molecule), a mast cell modulator, an anti-inflammatory agent (such as adrenocortical hormones, corticosteroids (such as betamethasone, budesonide, flunisolide, fluticasone, triamcinolone, methylprednisolone, prednisolone, prednisone, and hydrocortisone), glucocorticoids, steroids, nonsteroidal anti-inflammatory drugs (such as aspirin, ibuprofen, diclofenac sodium, and COX-2inhibitors), and leukotriene antagonists (such as montelukast, methylxanthine, zafirlukast, and zileuton), β2 agonists (such as salbutamol, biterol, fenoterol, isoetharine, orciprenaline, pirbuterol, salbutamol, terbutaline, formoterol, salmeterol, and salbutamol, terbutaline)), vitaxin, siplizumab, an antihistamine (such as diphenhydramine, meclastine, pyribenzamine, brompheniramine, chlorpheniramine, triprolidine, promethazine, hydroxyzine, astemizole, azatadine, cetirizine, cyproheptadine, loratadine, and fexofenadine), an anti-EphA2 antibody, and an active ingredient and extract of a common traditional Chinese medicine (such as anti-inflammatory and anti-cancer active small molecules and polysaccharide extracts (such as ginseng polysaccharides, dendrobium polysaccharides, Poria cocos polysaccharides, Ganoderma lucidum polysaccharides, Cordyceps polysaccharides, wolfberry polysaccharides, and Tremella fuciformis polysaccharides), and any combination thereof.

### EXAMPLES

The present application is further clarified through the following examples, but any or combinations of the examples should not be construed as limiting the scope or implementation of the present application. The scope of the present application is defined by the appended claims. In conjunction with the specification and the general knowledge of the art, those of ordinary skill in the art can clearly understand the scope defined by the claims. Those skilled in the art may make any modifications or changes to the technical solutions of the present application without deviating from the spirit and scope of the present application, and such modifications and changes are also included in the scope of the present application.

### Example 1 Preparation and Detection of Purified Antibodies

### 1. Preparation of Fc-IL-9 and IL-9-His Recombinant Proteins

A coding sequence for an IL-9 fragment (Glu19-Ile144) was synthesized, and Fc and His tags were added to an N-terminus and/or a C-terminus of the coding sequence, respectively. A resulting coding sequence was subcloned into eukaryotic expression vectors pcDNA3.4-His and pLVX-puro to construct antigen expression vectors pcDNA3.4-IL9 (19-144)-His (P15248) and pLVX-Fc2-IL9 (19-144)-Puro, respectively. The antigen expression vectors each were transiently transfected into 293F cells to produce recombinant proteins for immunization and antibody screening.

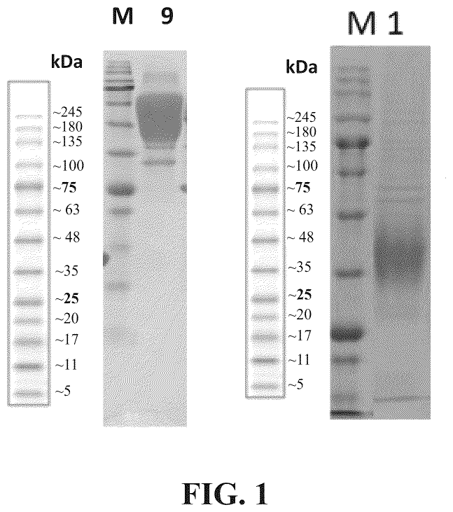

The purified Fc-IL-9 and IL-9-His recombinant proteins each were detected by SDS-PAGE. SDS-PAGE detection results (FIG. 1) show that the Fc-IL-9 recombinant protein has a size of about 40 kD, the IL-9-His recombinant protein has a size of about 30 kD, and purities of the Fc-IL-9 and IL-9-His recombinant proteins both are greater than 80%. Thus, the Fc-IL-9 and IL-9-His recombinant proteins can be used for immunization and titer detection.

### 2. Preparation of a CHO-S-IL-9 Cell Line (a CHO-S Cell Expressing IL-9 on the Cell Membrane)

A codon-optimized IL-9 sequence gene was synthesized and subcloned into a lentiviral expression vector Lenti-CMV-puro to prepare a target gene-overexpression lentiviral vector. A lentivirus was prepared with the constructed overexpression lentiviral vector and used to infect CHO-S cells, and a stable cell line was selected. The CHO-S cell line was incubated with an IL-9 positive control antibody 7F3com (see the patent CN1809383A). The expression of a target gene was detected by FACS. The 7F3com antibody has the following VH and VL sequences:

7F3com-VH
(SEQ ID NO: 34)
QVQLVQSGAEVKKPGSSVKVSCKASGGTFSYYWIEWVRQAPGQGLE
WMGEILPGSGTTNPNEKFKGRVTITADESTSTAYMELSSLRSEDTA
VYYCARADYYGSDYVKFDYWGQGTLVTVSS;
and
7F3com-VL
(SEQ ID NO: 35)
DIQMTQSPSSLSASVGDRVTITCKASQHVITHVTWYQQKPGKAPKL
LIYGTSYSYSGVPSRFSGSGSGTDFTLTISSLQPEDFATYYCQQFY
EYPLTFGGGTKVEIK.

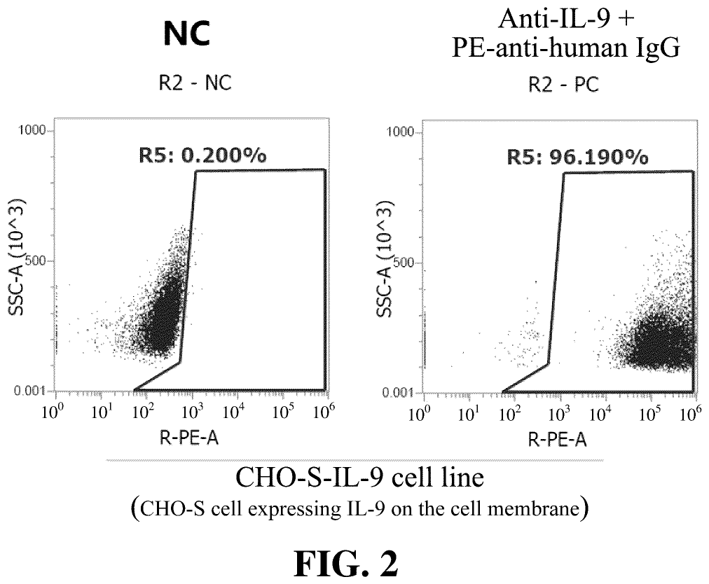

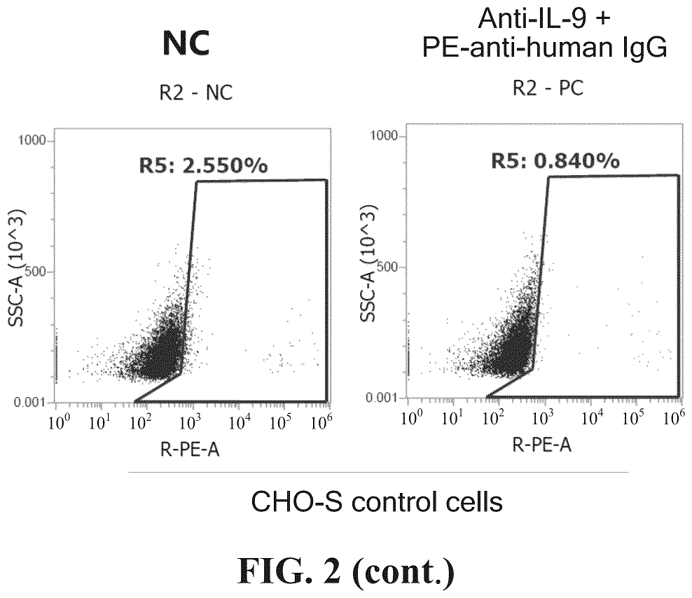

As shown in FIG. 2, the CHO-S-IL-9 cell line that could be used for immunization titer detection and antibody binding had been successfully selected.

### 3. Mouse Immunization and Detection

Through subcutaneous multi-point injection, ten 6 to 8-week-old female mice (which were at an SPF grade, of Balb/C and CD-1 lines with 5 in each, and marked 61 to 70) were immunized with the expressed Fc-IL-9 recombinant protein of 1 mg/mL at a dose of 100 μg/mouse. Three immunizations were conducted through subcutaneous injection on day 0, day 15, and day 30, respectively. After the immunizations, 10 μL of orbital blood was collected from each mouse and centrifuged to produce serum. The serum was serially diluted and then detected by ELISA. The serum was also diluted at 1:1,000 and then detected by FACS. According to ELISA and FACS detection results, mice 66 and 70 with prominent immunization titer detection results were selected for rush immunotherapy and hybridoma fusion through intraperitoneal injection.

### 4. Hybridoma Fusion and Screening

After undergoing 3 d of rush immunotherapy, mice were sacrificed, and mouse spleens were collected under sterile conditions to prepare single-cell suspensions of B cells. The single-cell suspensions of B cells each were mixed with non-secretory SP2/0 myeloma cells (Shanghai Cell Bank, Chinese Academy of Sciences, catalog number: TCM42), and cell fusion was conducted with a BTX cell electrofusion instrument. Merged cells were cultured in a complete medium for about 10 d, then the complete medium was replaced with an HT medium, and the merged cells were further cultured for 2 d. A resulting culture supernatant was collected for specific antibody detection.

Specifically, the culture supernatant collected was tested by ELISA for binding to IL-9-His. Titers of fusion clones were detected by FACS using CHO-S-IL-9 cells and CHO-S cells. Clones with strong binding were selected for subcloning. At the end of subcloning, ELISA and FACS were conducted for identification. The above process was repeated for a plurality of rounds until monoclones were produced. In the detection, NC was PBS, and PC was an immune serum diluted at 1:1,000. Finally, according to detection results, clones with high fluorescence values and prominent growth were selected to express and purify an antibody.

### 5. Detection of Purified Antibodies

### 5.1 ELISA Detection and EC₅₀ Counting of Purified Antibodies

ELISA was conducted according to a standard protocol to determine the affinity of a purified antibody for the IL-9-His recombinant protein. In short, the IL-9-His recombinant protein was diluted with sterile PBS and added at 100 μL/well to a 96-well plate. The plate was incubated at 4° C. overnight and then blocked with a blocking buffer. 100 μL of a purified anti-IL-9 antibody serially diluted was added to the plate, and the plate was incubated for 1 h at room temperature. PBS was added to a control well. The plate was washed with PBST 3 times. Then 100 μL of horseradish peroxidase (HRP)-labeled anti-mouse IgG H&L (BIODRAGON, CAT #BF03001) was added, and the plate was incubated for 1 h at room temperature. The plate was washed with PBST 3 times. Then a chromogenic reaction was conducted with a 3,3′,5,5′-tetramethylbenzidine (TMB) chromogenic solution at room temperature. An OD value in each well was read by a microplate reader. Data was processed with PRISM GraphPad, and curves were plotted. The EC₅₀ value for each candidate antibody was calculated. Table 1 shows EC₅₀ statistics of affinities of 44 purified antibodies for the IL-9-His recombinant protein.

TABLE 1
EC₅₀ statistics for purified antibodies

| Antibody No. | 19-D8-D3-B6 | 45-C9-B3-F8 | 19-C6-E9-G12 | 16-F12-D6-E2 | 37-D1-F9-C2 |
|---|---|---|---|---|---|
| EC50 | 0.03374 | 0.01797 | 0.01267 | 0.04438 | 0.05423 |
| Antibody No. | 47-G6-B6-E4 | 42-A3-C3-F2 | 47-D8-G8-B5 | 48-A9-A3-D7 | 16-A3-G9-B5 |
| EC50 | 0.03387 | 0.0341 | 0.08482 | 0.05498 | 0.06629 |
| Antibody No. | 48-A9-H11-C8 | 17-F5-E2-G2 | 46-G1-D3-B5 | 31-D6-B8-B8 | 37-A8-G12-D2 |
| EC50 | 0.1584 | 0.07831 | 0.1216 | 0.09616 | 0.3337 |
| Antibody No. | 42-A7-A4-F9 | 22-F2-E9-C8 | 46-G1-F6-C3 | 47-F12-B2-F9 | 20-B12-D8-B7 |
| EC50 | 0.07655 | 0.1387 | 0.3955 | 0.2126 | 0.9556 |
| Antibody No. | 1-H4-C1-C9 | 20-B12-A5-E11 | 43-C3-A10-F12 | 38-D8-C7-G9 | 8-D5-D1-E3 |
| EC50 | 0.05141 | 0.3626 | 0.03193 | 0.1761 | 0.0707 |

| Antibody No. | 29-B9-F5-B6 | 41-C12-D3-C5 | 17-A3-E1-D9 | 50-C12-B6-C6 |
|---|---|---|---|---|
| EC50 | 0.03499 | 0.07558 | 0.08403 | 0.02222 |
| Antibody No. | 42-G5-E3-C4 | 42-A3-C4-D2 | 4-F2-B1-D12 | 6-C10-H2-F6 |
| EC50 | 0.04156 | 0.1174 | 0.446 | 0.2426 |
| Antibody No. | 18-A12-G4-H5 | 45-G5-H6-D8 | 30-F5-B10-C4 | 3-C9-B8-D4 |
| EC50 | 0.07485 | 0.04299 | 0.5929 | 0.1539 |
| Antibody No. | 27-H6-B7-G9 | 17-A3-B7-C2 | 50-A3-F4-F4 | 31-B8-G4-F1 |
| EC50 | 0.4878 | 0.7355 | 0.4677 | 0.1907 |
| Antibody No. | 23-D10-H6-B1 | 23-C12-D2-D4 | 17-F7-F4-E7 | |
| EC50 | 0.103 | 0.1833 | 0.1828 | |

### 5.2 Detection of Blocking Activities of Purified Antibodies

A coding sequence for IL-9R was constructed in a eukaryotic expression vector pLVX-Hygro. 293F cells were infected with the constructed vector, and a 293-IL-9R cell line was selected for detection of blocking activity of antibodies. The IL-9 recombinant protein was incubated with IL-9R, and flow cytometry results showed that the IL-9 recombinant protein could specifically bind to IL-9R.

According to a standard protocol, the influence of purified antibodies on a binding ability of biotin-conjugated IL-9-Fc (Biotin-IL-9-Fc) to the 293-IL-9R recombinant cell line was detected by flow cytometry to determine blocking activities. In short, 293-IL-9R cells in a logarithmic growth phase were resuspended in PBS, adjusted to a cell density of 3×10⁶ cells/mL, and added to a 96-well U-shaped plate at 100 μL/well. A purified anti-IL-9 antibody to be tested of 20 μg/mL and Biotin-IL-9-Fc of 20 μg/mL were added to the plate, and the plate was incubated at 4° C. for 1 h. The plate was washed with PBS once. Then a secondary antibody (APC-Streptavidin, Biolegend®, CAT #405207) was added at 100 μL/well, and the plate was incubated at 4° C. for 45 min. Washing was conducted with PBS, and then resuspension was conducted with PBS. Flow cytometry was conducted. PC was the IL-9 positive antibody 7F3com.

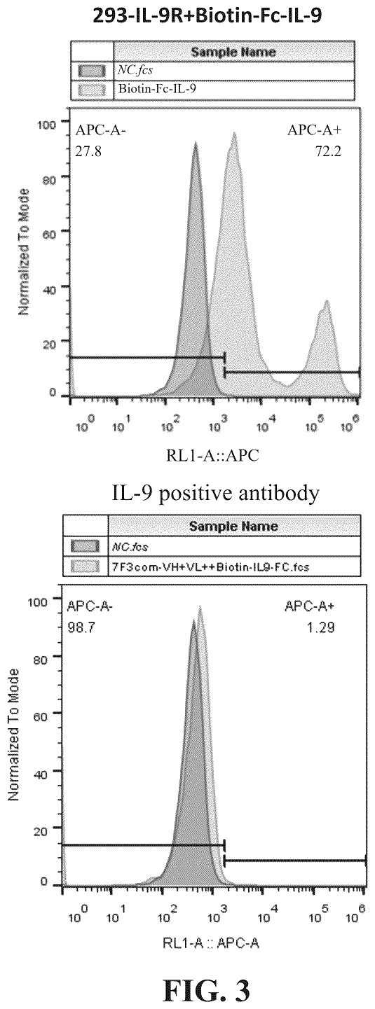

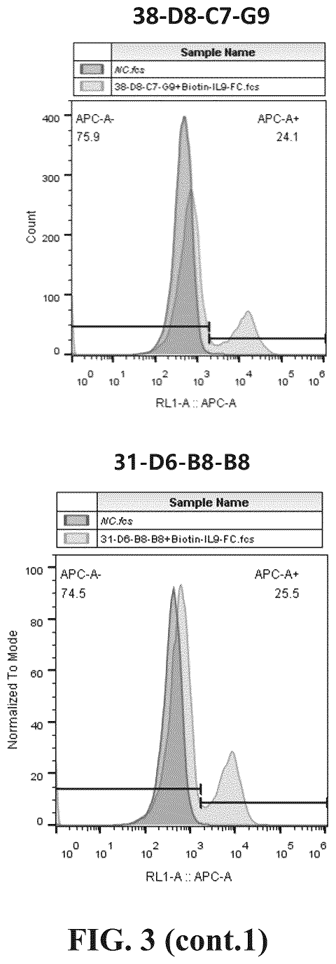

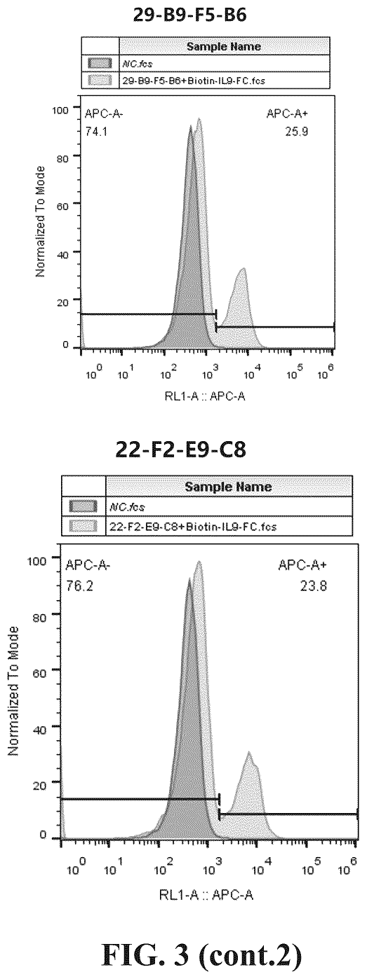

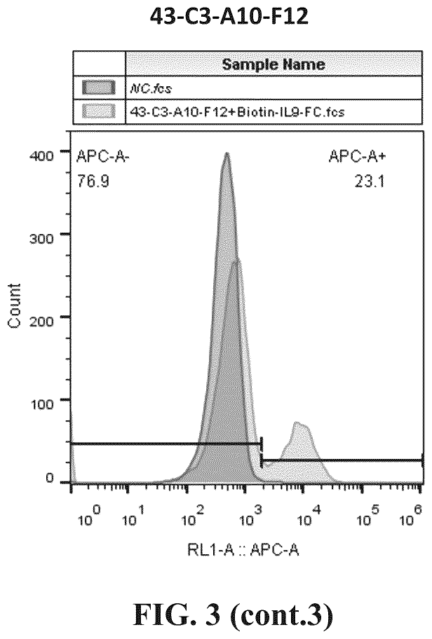

According to ELISA results and flow cytometry results of the blocking experiment (FIG. 3), the five purified antibodies 38-D8-C7-G9, 31-D6-B8-B8, 29-B9-F5-B6, 22-F2-E9-C8, and 43-C3-A10-F12 (which were re-numbered as A1 to A5, respectively) were selected for further hybridoma sequencing.

### 6. Hybridoma Sequencing

According to a standard protocol, total RNA was extracted from hybridoma cells and reverse-transcribed into a cDNA sample. VH and VL of an antibody were amplified by polymerase chain reaction (PCR) with hybridoma sequencing primers, and then TA cloning was conducted. Fragments produced by PCR were subcloned into a T vector, and clones were picked for sequencing. Hybridoma sequencing results were shown in Table 2 below, where A4 and A5 sequences were identical.

TABLE 2
CDR, VH, and VL sequences of A1 to A5 antibodies

| | | | SEQ ID |
|---|---|---|---|
| Antibody name | Region | Sequence | NO: |
| A1 | CDR-H1 | GYTFTGYW | 1 |
| (38-D8-C7-G9) | CDR-H2 | ILPGSGST | 2 |
| | CDR-H3 | ARRTYYYGIPEDY | 3 |
| | VH | EVQLKQSGAELMKPGASVKLSCKATGYTF | 4 |
| | | TGYWIEWVKLRPGHGLEWIGEILPGSGST | |
| | | NYNEEFKGKATFTADTSSNTAYMQLNSLT | |
| | | TEDSAIYYCARRTYYYGIPEDYWGQGTSV | |
| | | TVSS | |
| | CDR-L1 | QNVGTN | 5 |
| | CDR-L2 | SAS | |
| | CDR-L3 | QQYNSYPLT | 6 |
| | VL | DIVMTQSQKFMSTSVGDRVSVTCKASQN | 7 |
| | | VGTNVAWHQQKPGQSPKPLIYSASYRYSG | |
| | | VPDRFTGSGSGTDFTLTISNVQSEDLAEYF | |
| | | CQQYNSYPLTFGAGTKLELK | |
| A2 | CDR-H1 | GYSSTSGYY | 8 |
| (31-D6-B8-B8) | CDR-H2 | IRYDGSS | 9 |
| | CDR-H3 | ASYGITYDAMDY | 10 |
| | VH | EVQLKESGPGLVKPSQSLSLTCSVTGYSST | 11 |
| | | SGYYWNWIRQFPGNKLEWMGYIRYDGSS | |
| | | NHNPSLKNRISITRDTSKNQFFLKLNSVTT | |
| | | EDTATYYCASYGITYDAMDYWGQGTSVT | |
| | | VSS | |
| | CDR-L1 | ENIDSY | 12 |
| | CDR-L2 | AAT | |
| | CDR-L3 | QHYYSTPWT | 13 |
| | VL | DIQLTQPPASLSASVGETVTITCRASENIDS | 14 |
| | | YLAWYQQKQGKSPQLLVYAATLLADGVP | |
| | | SRFSGSGSGTQYSLKINSLQSEDVARYYCQ | |
| | | HYYSTPWTFGGGTKLELK | |
| A3 | CDR-H1 | GYTFSSYW | 15 |
| (29-B9-F5-B6) | CDR-H2 | ILPGSGSN | 16 |
| | CDR-H3 | AREGNYGEAWFAY | 17 |
| | VH | EVQLQESGAELMKPGASVKISCKATGYTF | 18 |
| | | SSYWIEWVKQRPGHGLEWIGQILPGSGSN | |
| | | NYNERFKGKATFTADTSSNTAYMQLSSLTS | |
| | | EDSAVYYCAREGNYGEAWFAYWGQGTLV | |
| | | TVSA | |
| | CDR-L1 | QSISNN | 19 |
| | CDR-L2 | YAS | |
| | CDR-L3 | QQSNSWPLT | 20 |
| | VL | DILLTQSPATLSVTPGDSVSLSCRASQSISN | 21 |
| | | NLHWYQQKSHESPRLLIKYASQSMSGIPSR | |
| | | FSGSGSGTDFTLSINSVETEDFGMYFCQQS | |
| | | NSWPLTFGAGTKLELK | |
| A4/A5 | CDR-H1 | GSSFTSGYY | 22 |
| (22-F2-E9-C8/ | CDR-H2 | IRYDGSN | 23 |
| 43-C3-A10-F12) | CDR-H3 | ASYGSSYDAMDY | 24 |
| | VH | EVQVVESGPGLVKPSQSLSLTCSVTGSSFT | 25 |
| | | SGYYWNWIRQFPGNKLEWMGYIRYDGSN | |
| | | NYNPSLKNRISITRDTSKNQFFLKLNSVTS | |
| | | EDTATYYCASYGSSYDAMDYWGQGTSVT | |
| | | VSS | |
| | CDR-L1 | ENIDSY | 12 |
| | CDR-L2 | AAT | |
| | CDR-L3 | QHYYSTPWT | 13 |
| | VI | DIQLTQPPASLSASVGETVTITCRASENIDS | 14 |
| | | YLAWYQQKQGKSPQLLVYAATLLADGVP | |
| | | SRFSGSGSGTQYSLKINSLQSEDVARYYCQ | |
| | | HYYSTPWTFGGGTKLELK | |

### Example 2 Preparation and Detection of Chimeric Antibodies

### 1. Construction and Verification of Chimeric Antibody Expression Vectors

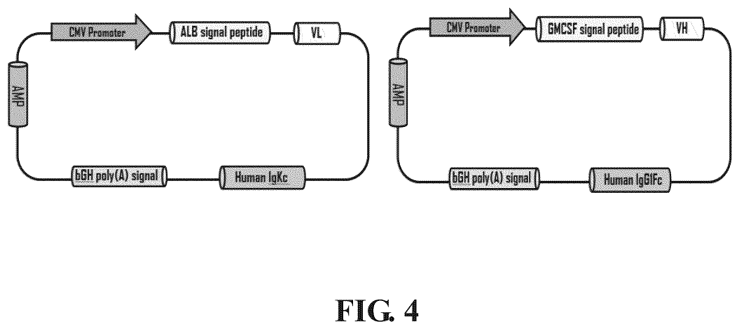

A VH coding sequence of an antibody produced from hybridoma sequencing was subcloned into a pcDNA3.4-IgG1 expression vector, and a VL coding sequence was subcloned into a pcDNA3.4-IgKc expression vector (FIG. 4). The constructed heavy-chain and light-chain expression vectors were transiently co-transfected into 293F cells (serum-free culture), and a resulting culture supernatant was collected for ELISA. A method for ELISA was the same as described in 5.1 of Example 1. Test results are shown in Table 3 below.

TABLE 3
Expression detection results of the chimeric antibodies
ELISA OD450

| | CA1 | CA2 | CA3 | CA4 |
|---|---|---|---|---|
| Antibody | (38-D8- | (31-D6- | (29-B9- | (22-F2- |
| concentration | C7-G9) | B8-B8) | F5-B6) | E9-C8) |
| 1 μg/mL | 2.429 | 2.661 | 2.707 | 2.628 |
| | 2.546 | 2.620 | 2.620 | 2.588 |
| 2 μg/mL | 2.482 | 2.486 | 2.560 | 2.451 |
| | 2.596 | 2.458 | 2.451 | 2.432 |
| PBS | 0.067 | 0.126 | 0.136 | 0.131 |
| | 0.244 | 0.163 | 0.141 | 0.144 |
| Blank | 0.067 | 0.073 | 0.074 | 0.071 |

The results showed that the four chimeric antibodies (CA1 to CA4) all could specifically bind to IL-9. The chimeric antibodies were further subjected to expression, purification, and blocking activity detection.

2. Expression, purification, and Blocking Activity Detection of Chimeric Antibodies

PBS was added at 2 mL/well to a 6-well plate, then the pcDNA3.4-IgG1 and pcDNA3.4-IgKc expression vectors were added, and thorough mixing was conducted by pipetting up and down. A LVTransm transfection reagent was added, and the plate was allowed to stand at room temperature for 10 min. A DNA/LVTransm complex was added to 293F cells, and thorough mixing was conducted. The cells were further cultured in a 37° C. and 5% CO₂ incubator. A culture supernatant was collected through centrifugation and filtered to produce a filtrate. The filtrate was transferred to a sterile centrifuge tube and loaded on a Protein A affinity column to purify a chimeric antibody.

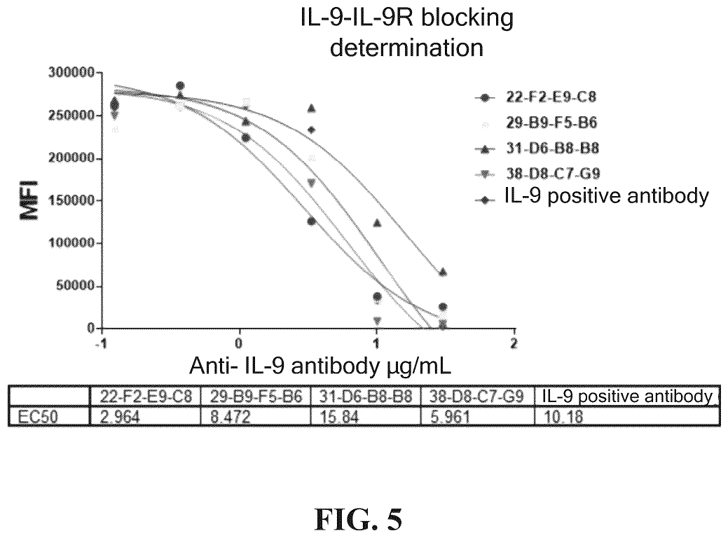

The chimeric antibody was serially diluted, and then the influence of the chimeric antibody on a binding ability of Biotin-IL-9-Fc to the 293-IL-9R recombinant cell line was detected by flow cytometry to determine a blocking activity. PC was an IL-9 positive antibody 7F3com. According to flow cytometry results (FIG. 5), the four chimeric antibodies CA1 to CA4 all exhibited a high blocking activity. In the following examples, a humanized antibody design was conducted with the 38-D8-C7-G9 chimeric antibody (CA1 antibody) as an example.

### Example 3 Construction and Detection of Humanized Antibodies

### 1. Humanized Antibody Design

According to the acquired amino acid sequence information of VH and VL of a murine antibody, a humanization design was conducted. With CDR sequences of the original antibody retained, different humanized antibody templates were selected for a heavy chain and a light chain according to results of germline alignment and results of antibody structure simulation. Candidate humanized antibody sequences were designed through reverse mutations for FRs after humanization (Table 4).

TABLE 4
Humanized antibody sequences

| | | SEQ |
|---|---|---|
| Region | Sequence | ID NO: |
| HM-VH1-IgG1 | EVQLKQSGAEVMKPGASVKVSCKATGYTFTGYWIEWVRLA | 26 |
| (VH1) | PGHGLEWMGEILPGSGSTNYNQKFQGRVTFTADTSINTAYM | |
| | ELSRLRSDDTAIYYCARRTYYYGIPEDYWGQGTLVTVSS | |
| HM-VH2-IgG1 | EVQLVQSGAEVMKPGASVKVSCKATGYTFTGYWIEWVRLA | 27 |
| (VH2) | PGQGLEWMGEILPGSGSTNYNQKFQGRVTFTADTSTNTAYM | |
| | ELSSLRSEDTAIYYCARRTYYYGIPEDYWGQGTLVTVSS | |
| HM-VH3-IgG1 | EVQLVQSGAEVMKPGASVKVSCKATGYTFTGYWIEWVRLA | 28 |
| (VH3) | PGQGLEWMGEILPGSGSTNYNQKLQGRVTFTADTSTNTAYM | |
| | ELRSLRSDDTAIYYCARRTYYYGIPEDYWGQGTLVTVSS | |
| HM-VH4-IgG1 | EVQLKQSGAEVMKPGASVKLSCKATGYTFTGYWIEWVKLA | 29 |
| (VH4) | PGHGLEWIGEILPGSGSTNYNEEFQGRVTFTADTSSNTAYME | |
| | LSRLRSDDTAIYYCARRTYYYGIPEDYWGQGTLVTVSS | |
| HM-VL1-IgKc | DIVMTQSQKFLSTSVGDRVTITCRASQNVGTNVAWHQQKPG | 30 |
| (VL1) | QSPKPLIYSASYRYSGVPSRFTGSGSGTDFTLTISNLQPEDFAE | |
| | YFCQQYNSYPLTFGAGTKLEIK | |
| HM-VL2-IgKc | DIVMTQSQKFFSTSTGDRVTITCRASQNVGTNVAWHQQKPG | 31 |
| (VL2) | QSPKPLIYSASYRYSGVPSRFTGSGSGTDFTLTISNLQSEDFAE | |
| | YFCQQYNSYPLTFGAGTKLEIK | |
| HM-VL3-IgKc | DIVMTQSPKFVSTSVGDRVTITCRASQNVGTNVAWHQQKPG | 32 |
| (VL3) | QSPKPLIYSASYRYSGVPSRFTGSGSGTDFTLTISNLQPEDFAE | |
| | YFCQQYNSYPLTFGAGTKLEIK | |
| HM-VL4-IgKc | DIVMTQSPKFLSASVGDRVTITCKASQNVGTNVAWHQQKPG | 33 |
| (VL4) | QSPKPLIYSASYRYSGVPSRFTGSGSGTDFTLTISNLQPEDFAE | |
| | YFCQQYNSYPLTFGAGTKLEIK | |

### 2. Gene Synthesis and Expression Vector Construction for Humanized Antibodies

Gene synthesis was conducted for heavy and light chains of each humanized antibody designed above. A heavy chain sequence was subcloned into a pcDNA3.4-IgG1 expression vector, and a light chain sequence was subcloned into a pcDNA3.4-IgKc expression vector (schematic diagrams of the vectors were shown in FIG. 4). After vectors were verified by sequencing to be correct, endotoxin-removed plasmids were prepared.

### 3. Expression and Detection of Humanized Antibodies

Humanized antibody expression vectors carrying a designed heavy-chain or light-chain sequence were transiently transfected into 293F cells in pairs, and then a resulting culture supernatant was collected. According to a standard protocol (with reference to part 5.1 in Example 1), with a human-mouse chimeric antibody CA1 constructed with VH and VL of the original murine antibody as a control, the binding of a humanized antibody to a target antigen was detected by ELISA. The method for ELISA was the same as described in 5.1 of Example 1. Results showed that the VH1+VL1, VH1+VL2, VH1+VL3, VH1+VL4, VH2+VL1, VH2+VL3, VH2+VL4, VH3+VL1, VH3+VL3, VH3+VL4, VH4+VL1, and VH4+VL3 combinations all could bind to IL-9 with high affinity, and the binding was basically the same as the binding of the chimeric antibodies. In the follow-up experiments, the four combinations of VH1−VL2, VH1−VL4, VH2−VL4, and VH3−VL4 (hu.CA1 to hu.CA4) were taken as examples for antibody purification and function testing.

### 4. Purification and Function Testing for Humanized Antibodies

The pcDNA3.4-IgG1 and pcDNA3.4-IgKc expression vectors corresponding to the selected antibody heavy/light chain combinations each were transfected into 293F cells with a LVTransm transfection reagent. The cells were cultured, and a resulting culture supernatant was collected through centrifugation. An antibody was purified with a Protein A affinity column. According to standard protocols, the affinity and blocking activity of a purified humanized antibody were detected by ELISA (with a 38-D8-C7-G9 (CA1) chimeric antibody and an IL-9 positive antibody 7F3com as controls) and flow cytometry (with 38-D8-C7-G9 (CA1) as a control), respectively.

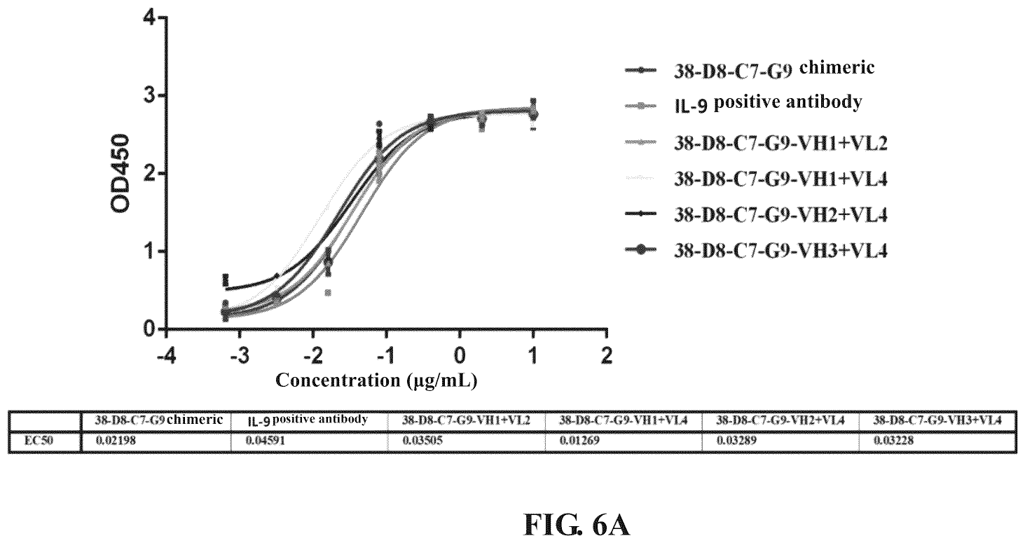

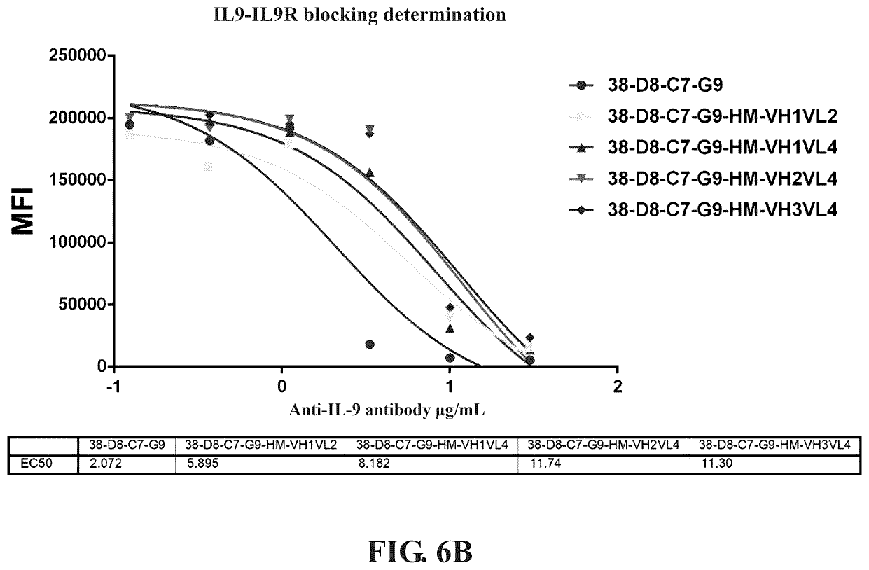

Results (FIG. 6A and FIG. 6B) showed that the humanized antibodies, the chimeric antibodies, and the positive control antibody exhibited consistent affinity. In the follow-up experiments, the affinity was detected with the two combinations of VH1−VL4 (hu.CA2) and VH1−VL2 (hu.CA1) as examples.

### 5. Affinity Detection for Humanized Antibodies

The affinity of a humanized antibody was detected by ForteBio (BLI). The chimeric (as a control) and humanized antibodies were immobilized with a Protein A sensor (SARTORIUS, Cat #18-5010) at an immobilization height of about 1 nm. An antigen protein sample was serially diluted with PBS to 125 nM, 62.5 nM, 31.25 nM, 15.625 nM, 7.81 nM, and 0 nM. An affinity detection procedure was as follows: equilibration for 60 s, binding for 180 s, and dissociation for 180 s.

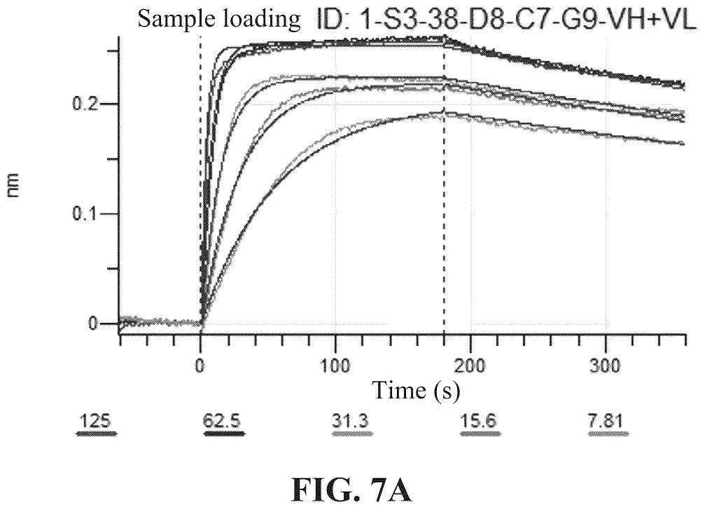

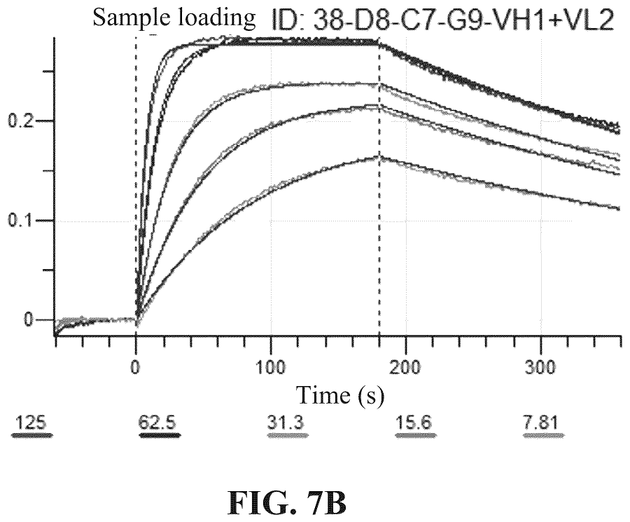

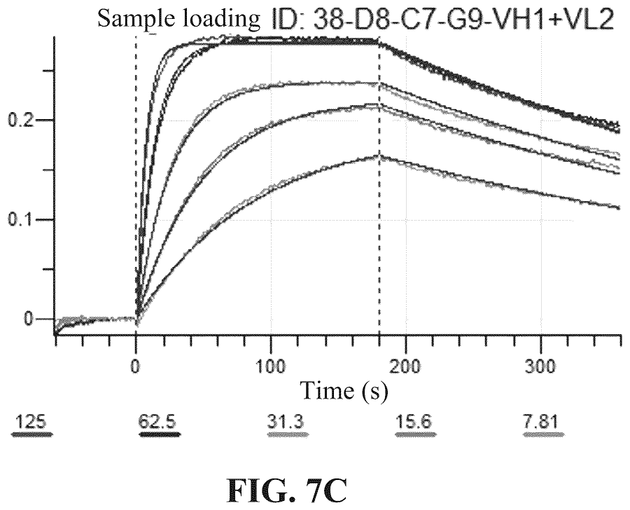

Affinity detection results of purified humanized antibodies were shown in FIG. 7A to FIG. 7C and Table 5 below. The results showed that a signal was concentration-dependent.

TABLE 5
Affinity detection results

| | Kon (M⁻¹s⁻¹) | Koff (s⁻¹) | KD (M) |
|---|---|---|---|
| Chimeric antibody | 2.13 × 10⁶ | 9.336 × 10⁻⁴ | 0.44 × 10⁻⁹ |
| VH1-VL4 | 1.165 × 10⁶ | 2.206 × 10⁻³ | 1.893 × 10⁻⁹ |
| VH1-VL2 | 1.066 × 10⁶ | 2.535 × 10⁻³ | 2.378 × 10⁻⁹ |

### Example 4 Treatment of a Mouse Pulmonary Fibrosis Model With an Anti-IL-9 Antibody

### Materials and Methods

6 to 8-week-old male C57BL/6J mice (purchased from Guangdong Sijiajingda Biotechnology Co., Ltd., license No.: SCXK (Guangdong) 2020-0052) were grouped, modeled, and administered according to the protocols shown in Tables 6 and 7 below. The modeling with bleomycin (Nippon Kayaku, 15 mg powder, and dose: 1.5 mg/kg) and silica (SiO₂, sigma, USA, S5631-500 g, and dose: 2.5 mg/mouse) was started on day 0 (DO). The administration was started on day 1 (D1), and normal saline was adopted as a solvent for an antibody. A positive drug pirfenidone (Beijing Kangdini Pharmaceutical Co., Ltd., 100 mg capsule) was adopted in a positive control group, and the humanized antibody hu.CA1 (VH1−VL2) of the present application was adopted in an anti-IL-9 antibody group.

TABLE 6
Bleomycin model group

| | Number of | | | |
|---|---|---|---|---|
| | animals | | Administration | Administration |
| Group | (mice) | Modeling | regimen | dose |
| Blank | 10 | Intratracheal | Normal saline | |
| control | | instillation | was | |
| group | | of PBS | intragastrically | |
| | | | administered | |
| | | | once a day | |
| | | | consecutively | |
| | | | for 21 d | |
| Negative | 10 | Intratracheal | Normal saline | |
| control | | instillation | was | |
| group | | of | intragastrically | |
| | | bleomycin | administered | |
| | | | once a day | |
| | | | consecutively | |
| | | | for 21 d | |
| Positive | 10 | Intratracheal | Pirfenidone was | 300 mg/kg |
| control | | instillation | intragastrically | |
| group | | of | administered | |
| | | bleomycin | once a day | |
| | | | consecutively | |
| | | | for 21 d | |
| Anti-IL-9 | 10 | Intratracheal | An anti-IL-9 | 100 μg |
| antibody | | instillation | antibody was | |
| | | of | intraperitoneally | |
| group | | bleomycin | injected every | |
| | | | other day | |

TABLE 7
Silica model group

| | Number | | | |
|---|---|---|---|---|
| | of animals | | Administration | Administration |
| Group | (mice) | Modeling | regimen | dose |
| Blank | 11 | Intratracheal | Normal saline was | |
| control | | instillation | intragastrically | |
| group | | of PBS | administered | |
| | | | once a day | |
| | | | consecutively | |
| | | | for 30 d | |
| Negative | 11 | Intratracheal | Normal saline was | |
| control | | instillation | intragastrically | |
| group | | of SiO₂ | administered | |
| | | | once a day | |
| | | | consecutively | |
| | | | for 30 d | |
| Positive | 11^{a} | Intratracheal | Pirfenidone was | 300 mg/kg |
| control | | instillation | intragastrically | |
| group | | of SiO₂ | administered | |
| | | | once a day | |
| | | | consecutively | |
| | | | for 30 d | |
| Anti-IL-9 | 11 | Intratracheal | An anti-IL-9 | 100 μg |
| antibody | | instillation | antibody was | |
| group | | of SiO₂ | intraperitoneally | |
| | | | injected every | |
| | | | other day | |

a: One animal died on D8 due to an incorrect intragastric operation, and at the end of the experiment, there were 10 animals in the corresponding group.

A body weight of each mouse was measured every day. After the bleomycin model group was administered for 21 d and the silica model group was administered for 30 d, mice were sacrificed to collect lung tissues. The lung tissues each were stained to detect pathological changes in the lung tissues. Specifically, a degree of alveolar inflammation was detected through HE staining (an HE staining kit was purchased from Shanghai Beyotime Biotechnology, C0105M). A degree of pulmonary fibrosis was detected through Masson staining (a Masson staining kit was purchased from Nanjing Jiancheng Institute of Biological Engineering, D026-1-2). Scoring criteria for the HE staining and Masson staining were shown in Table 8 and Table 9 below, respectively (as shown in references: Matute-Bello G, Downey G, Moore B B, et al. An official American Thoracic Society workshop report: features and measurements of experimental acute lung injury in animals. Am J Respir Cell Mol Biol. 2011; 44 (5): 725-738. doi: 10.1165/rcmb.2009-0210ST and Lee Hyun Ju, Goo Jin Mo, Kim Na Ra et al. Semiquantitative measurement of murine bleomycin-induced lung fibrosis in in vivo and postmortem conditions using microcomputed tomography: correlation with pathologic scores—initial results. [J].Invest Radiol, 2008, 43: 453-60).

TABLE 8
Scoring criteria for HE staining to determine inflammation

| | Score | | |
|---|---|---|---|
| Index | 0 | 0 | 2 |
| A. Neutrophils in | None | 1-5 | >5 |
| alveoli | | | |
| B. Neutrophils in | None | 1-5 | >5 |
| lung interstitium | | | |
| C. Transparent | None | 1 | >1 |
| membrane | | | |
| D. Protein fragments | None | 1 | >1 |
| filled in spaces | | | |
| E. Alveolar septum | <2X | 2X-4X | >4X |
| thickening | | | |

Score = [(20 * A) + (14 * B) + (7 * C) + (7 * D) + (2 * E)]/(field number * 100)

TABLE 9
Scoring criteria for Masson staining to determine fibrosis

| | Specific histopathological | |
|---|---|---|
| | characteristics | |
| Fibrosis score (0-20) | Interstitial fibrosis | 0-5 |
| | Honeycomb | 0-5 |
| | Metaplastic cells of alveolar | 0-2 |
| | walls line in air spaces | |
| | Interstitial smooth muscle | 0-2 |
| | Endomysium thickening of a | 0-2 |
| | blood vessel wall | |
| | Severity of bronchial wall | 0-2 |
| | fibrosis | |
| | Degree of bronchial wall fibrosis | 0-2 |

### Results

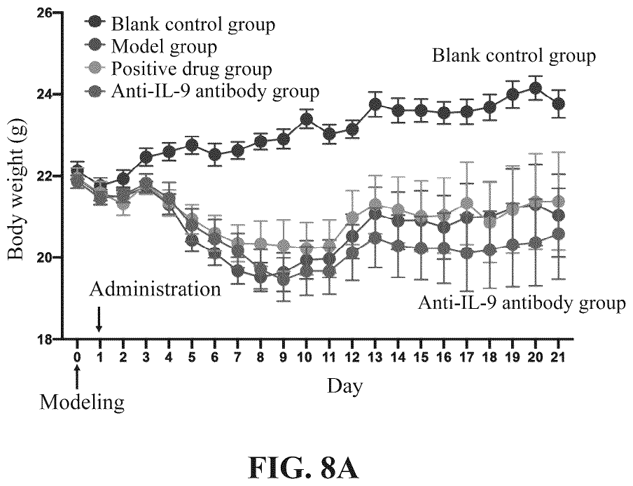

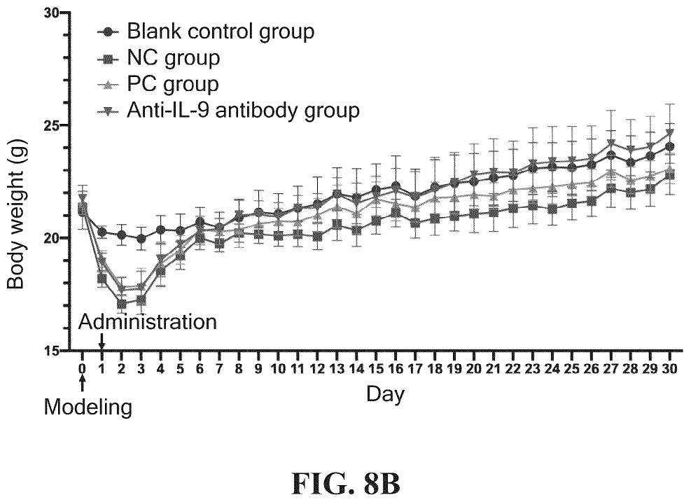

Body weights of the bleomycin mouse model group were shown in FIG. 8A. The results showed that body weights of mice in groups other than the blank control group all decreased after modeling, and rebounded on day 10 of administration (D10). During an administration process, body weights of the negative control group, the positive control group, and the anti-IL-9 antibody group changed in a same trend. Body weights of the silica mouse model group were shown in FIG. 8B. The results showed that body weights of mice in groups other than the blank control group all decreased after modeling, and rebounded from day 3 of administration (D3) and rebounded to the levels before modeling on day 12 of administration (D12). During an administration process, body weights of the negative control group, the positive control group, and the anti-IL-9 antibody group changed in a same trend. The above results indicate that the anti-IL-9 antibody has excellent safety.

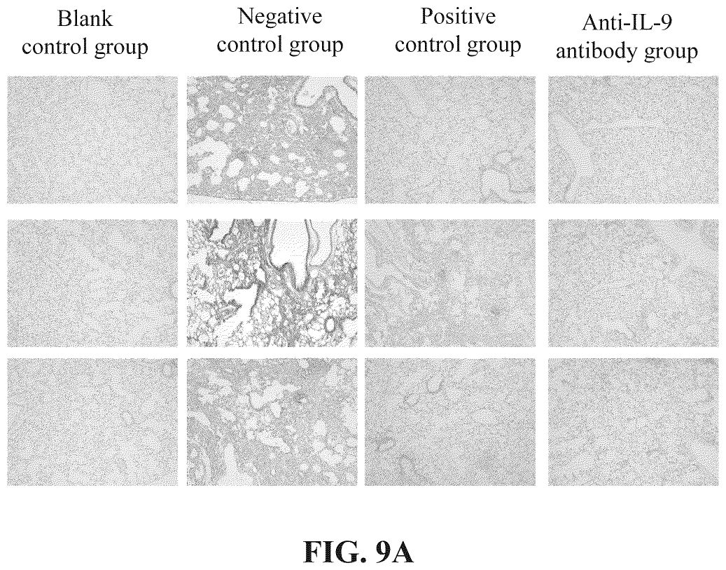

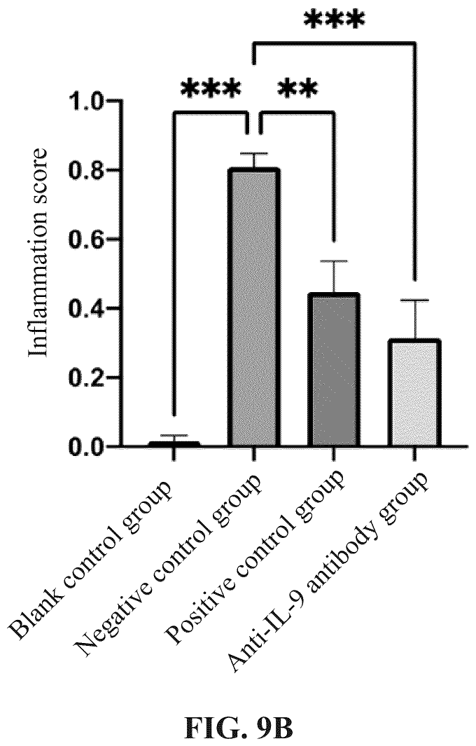

HE staining and inflammation scoring results of the bleomycin mouse model group were shown in FIG. 9A and FIG. 9B, respectively. The results showed that lung tissue structures of mice in the negative control group were seriously damaged, had many bleeding points, and underwent inflammatory cell infiltration. After medication, lung tissue structures of the positive control group and the anti-IL-9 antibody group all were improved, and bleeding points were reduced. Inflammation scoring results showed that both the anti-IL-9 antibody and the pirfenidone could significantly reduce an inflammation score, and an inflammation score of the anti-IL-9 antibody group was slightly lower than the pirfenidone group, indicating that the anti-IL-9 antibody exhibited a significant therapeutic effect for pulmonary fibrosis.

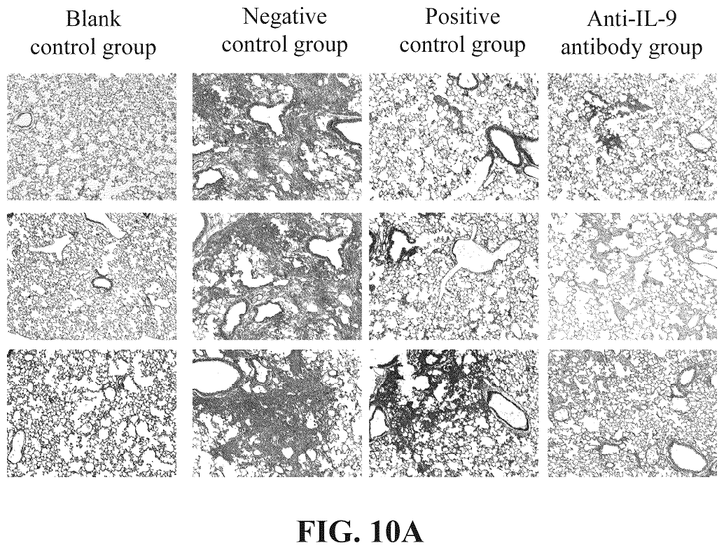

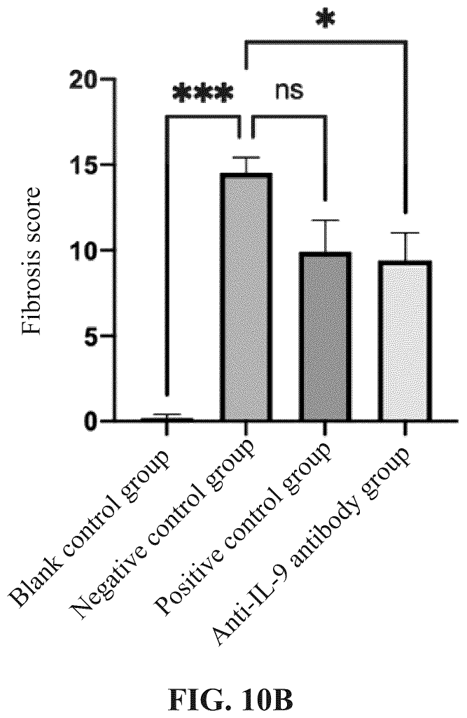

Masson staining and fibrosis scoring results of the bleomycin mouse model group were shown in FIG. 10A and FIG. 10B, respectively. The results showed that, compared with the blank control group, blue collagen fibers in lung tissues of mice in the negative control group significantly increased. After medication, collagen fibers and fibrosis regions of lung tissues in the positive control group and the anti-IL-9 antibody group were significantly reduced. Fibrosis scoring results showed that both the anti-IL-9 antibody and the positive drug pirfenidone could significantly reduce a fibrosis score, indicating that the anti-IL-9 antibody exhibited a significant therapeutic effect for pulmonary fibrosis.

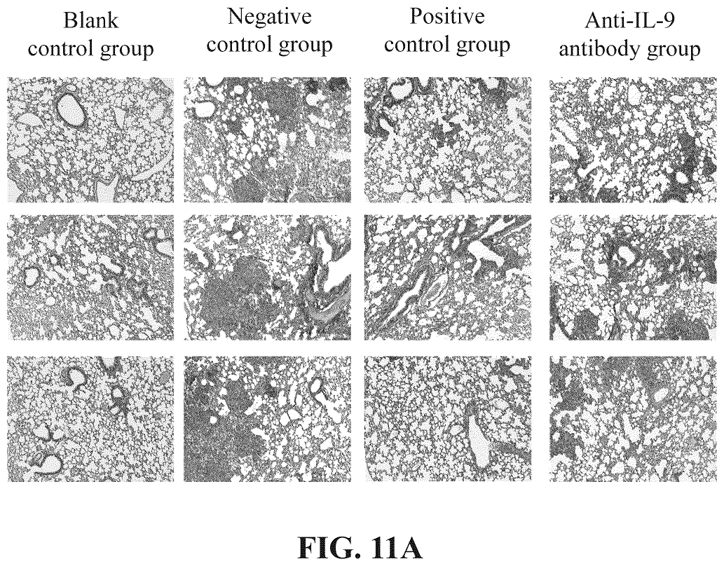

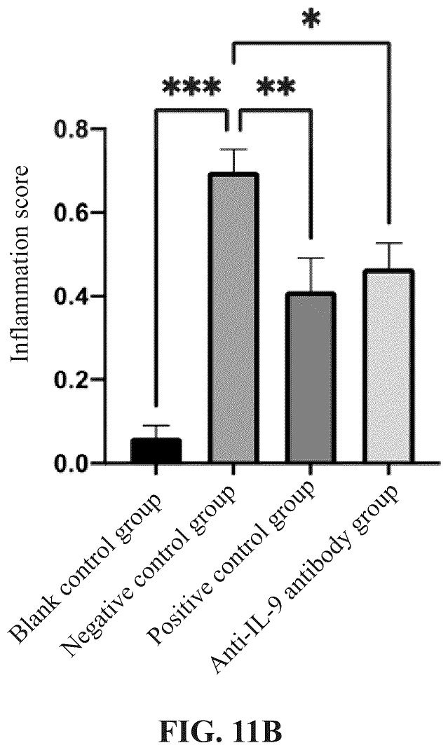

HE staining and inflammation scoring results of the silica mouse model group were shown in FIG. 11A and FIG. 11B, respectively. The results showed that lung tissues of mice in the negative control group underwent large inflammatory cell infiltration and serious structural damage, and had many bleeding points. After medication, lung tissue structures of the positive control group and the anti-IL-9 antibody group all were improved, and infiltration areas were reduced. Inflammation scoring results showed that both the anti-IL-9 antibody and the positive drug pirfenidone could significantly reduce an inflammation score, indicating that the anti-IL-9 antibody exhibited a significant therapeutic effect for pulmonary fibrosis.

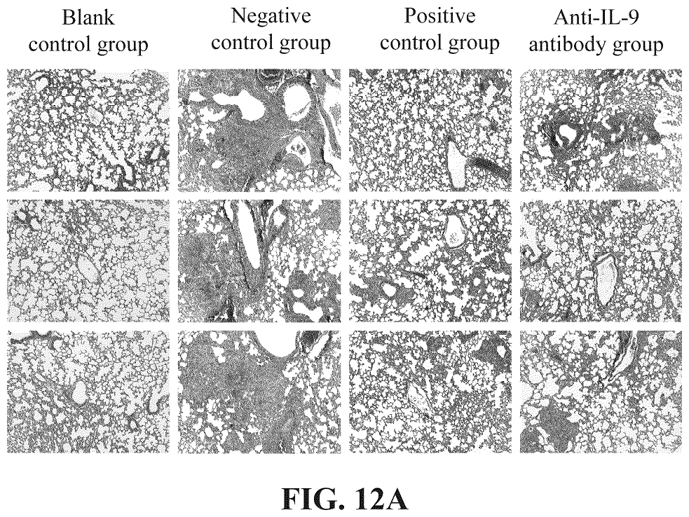

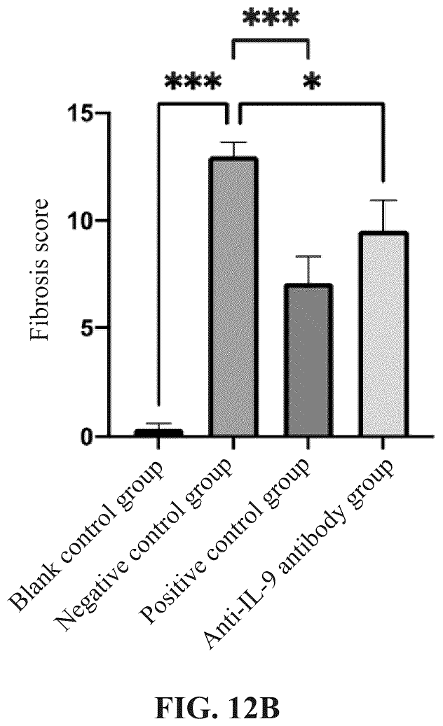

Masson staining and fibrosis scoring results of the silica mouse model group were shown in FIG. 12A and FIG. 12B, respectively. The results showed that, compared with the blank control group, in the negative control group, blue collagen fibers in lung tissues of mice significantly increased, and there were many bleeding points in lung tissues. After medication, collagen fibers and fibrosis regions of lung tissues in the positive control group and the anti-IL-9 antibody group were significantly reduced. Fibrosis scoring results showed that both the anti-IL-9 antibody and the positive drug pirfenidone could reduce a fibrosis score, indicating that the anti-IL-9 antibody exhibited a significant therapeutic effect for pulmonary fibrosis.

1. An antibody specifically binding to interleukin-9 (IL-9) or an antigen-binding fragment thereof, comprising a heavy chain variable region (VH) and a light chain variable region (VL), wherein
(i) the VH comprises CDR-H1, CDR-H2, and CDR-H3 having amino acid sequences set forth in SEQ ID NOs: 1-3, respectively, and the VL comprises CDR-L1, CDR-L2, and CDR-L3 having an amino acid sequence set forth in SEQ ID NO: 5, an amino acid sequence SAS, and an amino acid sequence set forth in SEQ ID NO: 6, respectively; or
(ii) the VH comprises CDR-H1, CDR-H2, and CDR-H3 having amino acid sequences set forth in SEQ ID NOs: 8-10, respectively, and the VL comprises CDR-L1, CDR-L2, and CDR-L3 having an amino acid sequence set forth in SEQ ID NO: 12, an amino acid sequence AAT, and an amino acid sequence set forth in SEQ ID NO: 13, respectively; or
(iii) the VH comprises CDR-H1, CDR-H2, and CDR-H3 having amino acid sequences set forth in SEQ ID NOs: 15-17, respectively, and the VL comprises CDR-L1, CDR-L2, and CDR-L3 having an amino acid sequence set forth in SEQ ID NO: 19, an amino acid sequence YAS, and an amino acid sequence set forth in SEQ ID NO: 20, respectively; or
(iv) the VH comprises CDR-H1, CDR-H2, and CDR-H3 having amino acid sequences set forth in SEQ ID NOs: 22-24, respectively, and the VL comprises CDR-L1, CDR-L2, and CDR-L3 having an amino acid sequence set forth in SEQ ID NO: 12, an amino acid sequence AAT, and an amino acid sequence set forth in SEQ ID NO: 13, respectively.

2. The antibody or the antigen-binding fragment thereof according to claim 1, wherein
(i) the VH comprises an amino acid sequence having at least 80%, at least 85%, at least 90%, at least 95%, at least 98%, at least 99%, or 100% sequence identity to SEQ ID NO: 4, and the VL comprises an amino acid sequence having at least 80%, at least 85%, at least 90%, at least 95%, at least 98%, at least 99%, or 100% sequence identity to SEQ ID NO: 7; or
(ii) the VH comprises an amino acid sequence having at least 80%, at least 85%, at least 90%, at least 95%, at least 98%, at least 99%, or 100% sequence identity to SEQ ID NO: 11, and the VL comprises an amino acid sequence having at least 80%, at least 85%, at least 90%, at least 95%, at least 98%, at least 99%, or 100% sequence identity to SEQ ID NO: 14; or
(iii) the VH comprises an amino acid sequence having at least 80%, at least 85%, at least 90%, at least 95%, at least 98%, at least 99%, or 100% sequence identity to SEQ ID NO: 18, and the VL comprises an amino acid sequence having at least 80%, at least 85%, at least 90%, at least 95%, at least 98%, at least 99%, or 100% sequence identity to SEQ ID NO: 21; or
(iv) the VH comprises an amino acid sequence having at least 80%, at least 85%, at least 90%, at least 95%, at least 98%, at least 99%, or 100% sequence identity to SEQ ID NO: 25, and the VL comprises an amino acid sequence having at least 80%, at least 85%, at least 90%, at least 95%, at least 98%, at least 99%, or 100% sequence identity to SEQ ID NO: 14.

3. The antibody or the antigen-binding fragment thereof according to claim 2, wherein
(i) the VH comprises an amino acid sequence set forth in SEQ ID NO: 4, and the VL comprises an amino acid sequence set forth in SEQ ID NO: 7; or
(ii) the VH comprises an amino acid sequence set forth in SEQ ID NO: 11, and the VL comprises an amino acid sequence set forth in SEQ ID NO: 14; or
(iii) the VH comprises an amino acid sequence set forth in SEQ ID NO: 18, and the VL comprises an amino acid sequence set forth in SEQ ID NO: 21; or
(iv) the VH comprises an amino acid sequence set forth in SEQ ID NO: 25, and the VL comprises an amino acid sequence set forth in SEQ ID NO: 14.

4. The antibody or the antigen-binding fragment thereof according to claim 1, wherein
the VH comprises an amino acid sequence having at least 80%, at least 85%, at least 90%, at least 95%, at least 98%, at least 99%, or 100% sequence identity to an amino acid sequence set forth in any one of SEQ ID NOs: 26-29, and the VL comprises an amino acid sequence having at least 80%, at least 85%, at least 90%, at least 95%, at least 98%, at least 99%, or 100% sequence identity to an amino acid sequence set forth in any one of SEQ ID NOs: 30-33.

5. The antibody or the antigen-binding fragment thereof according to claim 4, wherein
the VH comprises an amino acid sequence set forth in any one of SEQ ID NOs: 26-29, and the VL comprises an amino acid sequence set forth in any one of SEQ ID NOs: 30-33.

6. The antibody or the antigen-binding fragment thereof according to claim 4, wherein
(i) the VH comprises an amino acid sequence set forth in SEQ ID NO: 26, and the VL comprises an amino acid sequence set forth in SEQ ID NO: 31; or
(ii) the VH comprises an amino acid sequence set forth in SEQ ID NO: 26, and the VL comprises an amino acid sequence set forth in SEQ ID NO: 33; or
(iii) the VH comprises an amino acid sequence set forth in SEQ ID NO: 27, and the VL comprises an amino acid sequence set forth in SEQ ID NO: 33; or
(iv) the VH comprises an amino acid sequence set forth in SEQ ID NO: 28, and the VL comprises an amino acid sequence set forth in SEQ ID NO: 33.

7. The antibody or the antigen-binding fragment thereof according to claim 1, wherein the antibody is selected from the group consisting of IgG, IgA, IgM, IgE, and IgD isotypes, and the IgG isotype is selected from the group consisting of IgG1, IgG2, IgG3, and IgG4 subtypes.

8. The antibody or the antigen-binding fragment thereof according to claim 1, wherein the antibody is selected from the group consisting of a murine antibody, a chimeric antibody, a humanized antibody, and a fully human antibody.

9. The antibody or the antigen-binding fragment thereof according to claim 1, wherein the antigen-binding fragment is selected from the group consisting of Fab, Fab′, F(ab′)₂, Fv, scFv, and ds-scFv.

10. The antibody or the antigen-binding fragment thereof according to claim 1, wherein the antibody is a bispecific antibody or a multispecific antibody.

11. A nucleic acid comprising a nucleotide sequence encoding the antibody or the antigen-binding fragment thereof according to claim 1.

12. A vector comprising a nucleotide sequence encoding the antibody or the antigen-binding fragment thereof according to claim 1.

13. A host cell comprising the nucleic acid according to claim 11.

14. A host cell comprising the vector according to claim 12.

15. A pharmaceutical composition, comprising: the antibody or the antigen-binding fragment thereof according to claim 1, and a pharmaceutically acceptable carrier or excipient.

16. The pharmaceutical composition according to claim 15, further comprising a second therapeutic agent.

17. The pharmaceutical composition according to claim 16, wherein the second therapeutic agent is selected from the group consisting of an antibody, a chemotherapeutic agent, and a small molecule drug.

18. The pharmaceutical composition according to claim 16, wherein the second therapeutic agent is selected from the group consisting of an antiviral agent, an antibacterial agent, an antifungal agent, an antiangiogenic agent, a TNF-α antagonist, an immunomodulator, an anticancer agent, a mast cell modulator, an anti-inflammatory agent, an antihistamine, and an active ingredient and extract of a common traditional Chinese medicine, and a combination thereof.

19. A method for preventing or treating a disease associated with abnormal expression of IL-9 in a subject, comprising administering a therapeutically effective amount of the antibody or the antigen-binding fragment thereof according to claim 1 to the subject.

20. A method for preventing or treating a disease associated with abnormal expression of IL-9 in a subject, comprising administering a therapeutically effective amount of the pharmaceutical composition according to claim 15 to the subject.

21. A method for preventing or treating a disease associated with abnormal expression of IL-9 in a subject, comprising administering a therapeutically effective amount of the pharmaceutical composition according to claim 17 to the subject.

22. A method for preventing or treating a disease associated with abnormal expression of IL-9 in a subject, comprising administering a therapeutically effective amount of the pharmaceutical composition according to claim 18 to the subject.

23. The method according to claim 19, wherein the disease is a respiratory disease, and the respiratory disease is selected from the group consisting of acute infectious pneumonia, hypersensitivity pneumonitis, bronchial asthma, chronic obstructive pulmonary disease, and pulmonary fibrosis.

24. The method according to claim 19, wherein the disease is an autoimmune disease, and the autoimmune disease is selected from the group consisting of systemic lupus erythematosus, rheumatoid arthritis, systemic vasculitis, scleroderma, dermatomyositis, mixed connective tissue disease, allergic rhinitis, allergic conjunctivitis, autoimmune hemolytic anemia, autoimmune thyroid disease, ulcerative colitis, Sjogren's syndrome, psoriatic arthritis, multiple sclerosis, chronic lymphocytic thyroiditis, insulin-dependent diabetes mellitus, myasthenia gravis, pernicious anemia with chronic atrophic gastritis, pulmonary hemorrhage-nephritic syndrome, pemphigus vulgaris, pemphigoid, multiple cerebrospinal sclerosis, acute idiopathic polyneuritis, and autoimmune adrenal insufficiency.

25. The method according to claim 19, wherein the disease is a hematologic cancer, and the hematologic cancer is selected from the group consisting of leukemia, malignant plasma cell neoplasm, and lymphoma.

26. The method according to claim 25, wherein the hematologic cancer is selected from the group consisting of chronic myeloid leukemia, acute myeloid leukemia, chronic lymphocytic leukemia, acute lymphocytic leukemia, multiple myeloma, monoclonal gammopathy of undetermined significance (MGUS), non-Hodgkin's lymphoma, and Hodgkin's lymphoma.

27. The method according to claim 19, further comprising administering a second therapeutic agent to the subject.

28. The method according to claim 27, wherein the second therapeutic agent is selected from the group consisting of an antiviral agent, an antibacterial agent, an antifungal agent, an antiangiogenic agent, a TNF-α antagonist, an immunomodulator, an anticancer agent, a mast cell modulator, an anti-inflammatory agent, an antihistamine, and an active ingredient and extract of a common traditional Chinese medicine, and a combination thereof.


SEQUENCE LISTING (35 sequences)

SEQ ID NO: 1 - protein, 8 aa, synthetic construct
GYTFTGYW

SEQ ID NO: 2 - protein, 8 aa, synthetic construct
ILPGSGST

SEQ ID NO: 3 - protein, 13 aa, synthetic construct
ARRTYYYGIPEDY

SEQ ID NO: 4 - protein, 120 aa, synthetic construct
EVQLKQSGAELMKPGASVKLSCKATGYTFTGYWIEWVKLRPGHGLEWIGEILPGSGSTNYNEEFKGKATFTADTSSNTAYMQLNSLTTEDSAIYYCARRTYYYGIPEDYWGQGTSVTVSS

SEQ ID NO: 5 - protein, 6 aa, synthetic construct
QNVGTN

SEQ ID NO: 6 - protein, 9 aa, synthetic construct
QQYNSYPLT

SEQ ID NO: 7 - protein, 107 aa, synthetic construct
DIVMTQSQKFMSTSVGDRVSVTCKASQNVGTNVAWHQQKPGQSPKPLIYSASYRYSGVPDRFTGSGSGTDFTLTISNVQSEDLAEYFCQQYNSYPLTFGAGTKLELK

SEQ ID NO: 8 - protein, 9 aa, synthetic construct
GYSSTSGYY

SEQ ID NO: 9 - protein, 7 aa, synthetic construct
IRYDGSS

SEQ ID NO: 10 - protein, 12 aa, synthetic construct
ASYGITYDAMDY

SEQ ID NO: 11 - protein, 119 aa, synthetic construct
EVQLKESGPGLVKPSQSLSLTCSVTGYSSTSGYYWNWIRQFPGNKLEWMGYIRYDGSSNHNPSLKNRISITRDTSKNQFFLKLNSVTTEDTATYYCASYGITYDAMDYWGQGTSVTVSS

SEQ ID NO: 12 - protein, 6 aa, synthetic construct
ENIDSY

SEQ ID NO: 13 - protein, 9 aa, synthetic construct
QHYYSTPWT

SEQ ID NO: 14 - protein, 107 aa, synthetic construct
DIQLTQPPASLSASVGETVTITCRASENIDSYLAWYQQKQGKSPQLLVYAATLLADGVPSRFSGSGSGTQYSLKINSLQSEDVARYYCQHYYSTPWTFGGGTKLELK

SEQ ID NO: 15 - protein, 8 aa, synthetic construct
GYTFSSYW

SEQ ID NO: 16 - protein, 8 aa, synthetic construct
ILPGSGSN

SEQ ID NO: 17 - protein, 13 aa, synthetic construct
AREGNYGEAWFAY

SEQ ID NO: 18 - protein, 120 aa, synthetic construct
EVQLQESGAELMKPGASVKISCKATGYTFSSYWIEWVKQRPGHGLEWIGQILPGSGSNNYNERFKGKATFTADTSSNTAYMQLSSLTSEDSAVYYCAREGNYGEAWFAYWGQGTLVTVSA

SEQ ID NO: 19 - protein, 6 aa, synthetic construct
QSISNN

SEQ ID NO: 20 - protein, 9 aa, synthetic construct
QQSNSWPLT

SEQ ID NO: 21 - protein, 107 aa, synthetic construct
DILLTQSPATLSVTPGDSVSLSCRASQSISNNLHWYQQKSHESPRLLIKYASQSMSGIPSRFSGSGSGTDFTLSINSVETEDFGMYFCQQSNSWPLTFGAGTKLELK

SEQ ID NO: 22 - protein, 9 aa, synthetic construct
GSSFTSGYY

SEQ ID NO: 23 - protein, 7 aa, synthetic construct
IRYDGSN

SEQ ID NO: 24 - protein, 12 aa, synthetic construct
ASYGSSYDAMDY

SEQ ID NO: 25 - protein, 119 aa, synthetic construct
EVQVVESGPGLVKPSQSLSLTCSVTGSSFTSGYYWNWIRQFPGNKLEWMGYIRYDGSNNYNPSLKNRISITRDTSKNQFFLKLNSVTSEDTATYYCASYGSSYDAMDYWGQGTSVTVSS

SEQ ID NO: 26 - protein, 120 aa, synthetic construct
EVQLKQSGAEVMKPGASVKVSCKATGYTFTGYWIEWVRLAPGHGLEWMGEILPGSGSTNYNQKFQGRVTFTADTSINTAYMELSRLRSDDTAIYYCARRTYYYGIPEDYWGQGTLVTVSS

SEQ ID NO: 27 - protein, 120 aa, synthetic construct
EVQLVQSGAEVMKPGASVKVSCKATGYTFTGYWIEWVRLAPGQGLEWMGEILPGSGSTNYNQKFQGRVTFTADTSTNTAYMELSSLRSEDTAIYYCARRTYYYGIPEDYWGQGTLVTVSS

SEQ ID NO: 28 - protein, 120 aa, synthetic construct
EVQLVQSGAEVMKPGASVKVSCKATGYTFTGYWIEWVRLAPGQGLEWMGEILPGSGSTNYNQKLQGRVTFTADTSTNTAYMELRSLRSDDTAIYYCARRTYYYGIPEDYWGQGTLVTVSS

SEQ ID NO: 29 - protein, 120 aa, synthetic construct
EVQLKQSGAEVMKPGASVKLSCKATGYTFTGYWIEWVKLAPGHGLEWIGEILPGSGSTNYNEEFQGRVTFTADTSSNTAYMELSRLRSDDTAIYYCARRTYYYGIPEDYWGQGTLVTVSS

SEQ ID NO: 30 - protein, 107 aa, synthetic construct
DIVMTQSQKFLSTSVGDRVTITCRASQNVGTNVAWHQQKPGQSPKPLIYSASYRYSGVPSRFTGSGSGTDFTLTISNLQPEDFAEYFCQQYNSYPLTFGAGTKLEIK

SEQ ID NO: 31 - protein, 107 aa, synthetic construct
DIVMTQSQKFFSTSTGDRVTITCRASQNVGTNVAWHQQKPGQSPKPLIYSASYRYSGVPSRFTGSGSGTDFTLTISNLQSEDFAEYFCQQYNSYPLTFGAGTKLEIK

SEQ ID NO: 32 - protein, 107 aa, synthetic construct
DIVMTQSPKFVSTSVGDRVTITCRASQNVGTNVAWHQQKPGQSPKPLIYSASYRYSGVPSRFTGSGSGTDFTLTISNLQPEDFAEYFCQQYNSYPLTFGAGTKLEIK

SEQ ID NO: 33 - protein, 107 aa, synthetic construct
DIVMTQSPKFLSASVGDRVTITCKASQNVGTNVAWHQQKPGQSPKPLIYSASYRYSGVPSRFTGSGSGTDFTLTISNLQPEDFAEYFCQQYNSYPLTFGAGTKLEIK

SEQ ID NO: 34 - protein, 122 aa, synthetic construct
QVQLVQSGAEVKKPGSSVKVSCKASGGTFSYYWIEWVRQAPGQGLEWMGEILPGSGTTNPNEKFKGRVTITADESTSTAYMELSSLRSEDTAVYYCARADYYGSDYVKFDYWGQGTLVTVSS

SEQ ID NO: 35 - protein, 107 aa, synthetic construct
DIQMTQSPSSLSASVGDRVTITCKASQHVITHVTWYQQKPGKAPKLLIYGTSYSYSGVPSRFSGSGSGTDFTLTISSLQPEDFATYYCQQFYEYPLTFGGGTKVEIK